(19) United States
(12) Patent Application Publication
(10) Pub. No.: US 2013/0046083 A1
(43) Pub. Date: Feb. 21, 2013

(54) OLIGONUCLEOTIDE LIGATION

(71) Applicants: Tom Brown, Southampton (GB); Afaf Helmy El-Sagheer, Southampton (GB)

(72) Inventors: Tom Brown, Southampton (GB); Afaf Helmy El-Sagheer, Southampton (GB)

(21) Appl. No.: 13/210,948

(22) Filed: Aug. 16, 2011

(51) Int. Cl. C07H 21/00; C07H 1/00

(52) U.S. Cl. 536/23.1; 536/25.3

(57) ABSTRACT

Oligonucleotide chemistry is central to the advancement of core technologies such as DNA sequencing, forensic and genetic analysis and has impacted greatly on the discipline of molecular biology. Oligonucleotides and their analogues are essential tools in these areas. They are often produced by automated solid-phase phosphoramidite synthesis but it is difficult to synthesize long DNA and RNA sequences by this method. Methods are proposed for ligating oligonucleotides together, in particular the use of an azide-alkyne coupling reaction to ligate the backbones of oligonucleotides together to form longer oligonucleotides than can be synthesized using current phosphoramidite synthesis methods.

### FIELD OF THE INVENTION

This invention relates to a method for ligating oligonucleotides together, in particular it relates to use of an azide-alkyne coupling reaction to ligate the backbones of oligonucleotides together. It also relates to oligonucleotides comprising a triazole phosphodiester mimic.

All publications referred to in this application are hereby incorporated by reference in their entirety.

### BACKGROUND OF THE INVENTION

Oligonucleotide chemistry is central to the advancement of core technologies such as DNA sequencing, forensic and genetic analysis and has impacted greatly on the discipline of molecular biology. Oligonucleotides and their analogues are essential tools in these areas. They are often produced by automated solid-phase phosphoramidite synthesis. However, this process can only assemble DNA strands up to about 150 bases in length. Synthesis of long RNA strands is even more difficult owing to problems caused by the presence of the 2′-hydroxyl group of ribose which requires selective protection during oligonucleotide assembly. This reduces the coupling efficiency of RNA phosphoramidite monomers due to steric hindrance. In addition, side-reactions which occur during the removal (or premature loss) of the 2′-protecting groups cause phosphodiester backbone cleavage and 3′ to 2′ phosphate migration. Although several ingenious strategies have been developed to minimize these problems and to improve the synthesis of long RNA molecules, the chemical complexity of solid-phase RNA synthesis dictates that constructs longer than 50 nucleotides in length remain difficult to prepare. Most biologically important DNA and RNA molecules for example genes, ribozymes, aptamers and riboswitches are significantly longer than what is currently achievable by solid-phase synthesis, so new approaches to the synthesis of long DNA and RNA molecules are urgently required.

Although DNA and RNA synthesis by enzymatic replication or transcription might seem a viable alternative, it does not permit the site-specific incorporation of multiple modifications at sugars, bases, or phosphates and also leads to the loss of epigenetic information such as DNA methylation.

In contrast, automated solid-phase DNA and RNA synthesis is compatible with the introduction of methylated nucleotides, fluorescent tags, isotopic labels (for NMR studies) and other groups to improve biological activity and resistance to enzymatic degradation. The scope and utility of important DNA and RNA constructs can be significantly extended by such chemical modifications.

Another drawback of enzymatic replication or transcription is that the DNA and RNA products can only be cost-effectively produced at a small scale. The scale of chemical synthesis, by contrast, is potentially unlimited.

Previous studies have attempted to chemically ligate synthesized oligonucleotides to form longer DNA molecules as described in WO2008/120016, Kumar et al. 2007, J Am Chem Soc 129, 6859-6864, Kocalka et al. 2008, Chem Bio Chem, 9, 1280-1285, and El-Sagheer et al. 2009, J Am Chem Soc. 131(11), 3958-3964. The drawback with these molecules was that, because they contained unnatural linkages between the oligonucleotides, they were not fully active in a biological system. DNA and RNA polymerases could not read these nucleotide sequences accurately and mis-read or missed out nucleotides when trying to replicate the sequences.

Enzymatic ligation using, for example T4 DNA ligase can be used to join oligonucleotides but the use of ligases has other drawbacks; they are often contaminated with RNases which can partially degrade the ligation products, and the ligation protocols require removal of the ligase protein to produce pure DNA or RNA. Moreover, enzymatic ligation methods are not suitable for the large scale synthesis of DNA or RNA, and the yields of enzymatic ligation are sometimes low, particularly when using chemically modified DNA or RNA substrates or mixed DNA/RNA strands.

It would therefore be advantageous to provide a method that can be used on an industrial scale and can synthesize long DNA and RNA molecules that can be read correctly by DNA and RNA polymerases and hence can be used for in vitro and in vivo applications including applications in biology and nanotechnology.

### SUMMARY OF THE INVENTION

It is an aim of embodiments of the present invention to provide an efficient method of chemical ligation that can ligate oligonucleotides together by forming a link between the oligonucleotide backbones that is a triazole phosphodiester mimic and can be read through by DNA and RNA polymerases as described in El-Sagheer and Brown 2010, PNAS vol. 107 no. 35, 15329-15334 and El-Sagheer et al. 2011, PNAS vol. 108 no. 28. 11338-11343, which are incorporated herein in their entirety.

According to a first aspect of the present invention a method is provided for ligating one or more oligonucleotides together. The method comprises reacting at least one alkyne group with at least one azide group to form at least one triazole phosphodiester mimic. The reaction is selected from the following reaction schemes. The reaction schemes are drawn showing a linkage being formed between deoxyribose groups, for example of a DNA molecule. Equivalent reactions can be carried out between the ribose groups of an RNA molecule and therefore the RNA equivalents of the below reactions, linking the ribose groups of an RNA molecule are also contemplated within the scope of the present invention. The reactions are also applicable to nucleic acid analogues containing modifications to the sugars, for example 2′-O methyl RNA, 2′-fluoro RNA and/or LNA.

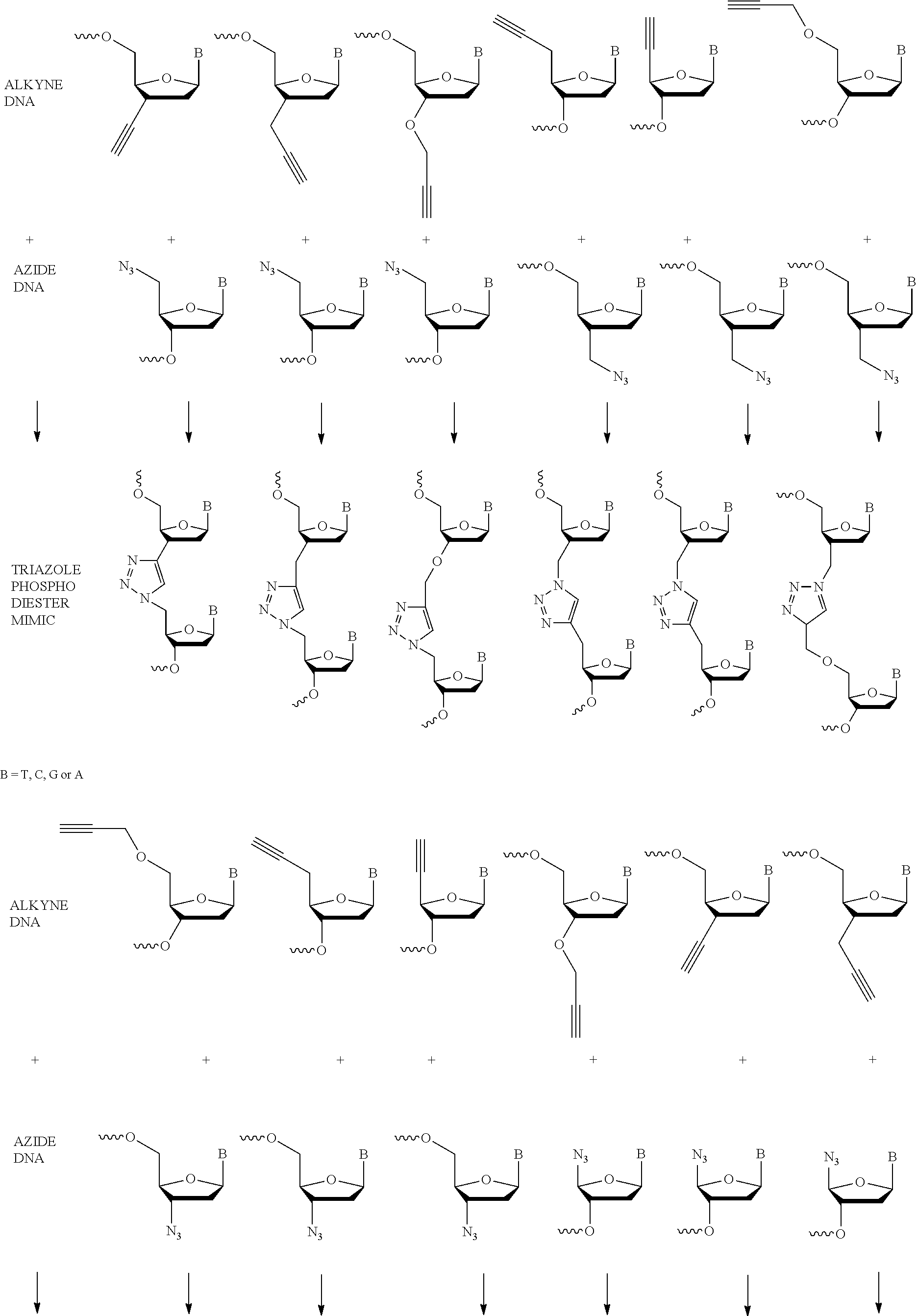

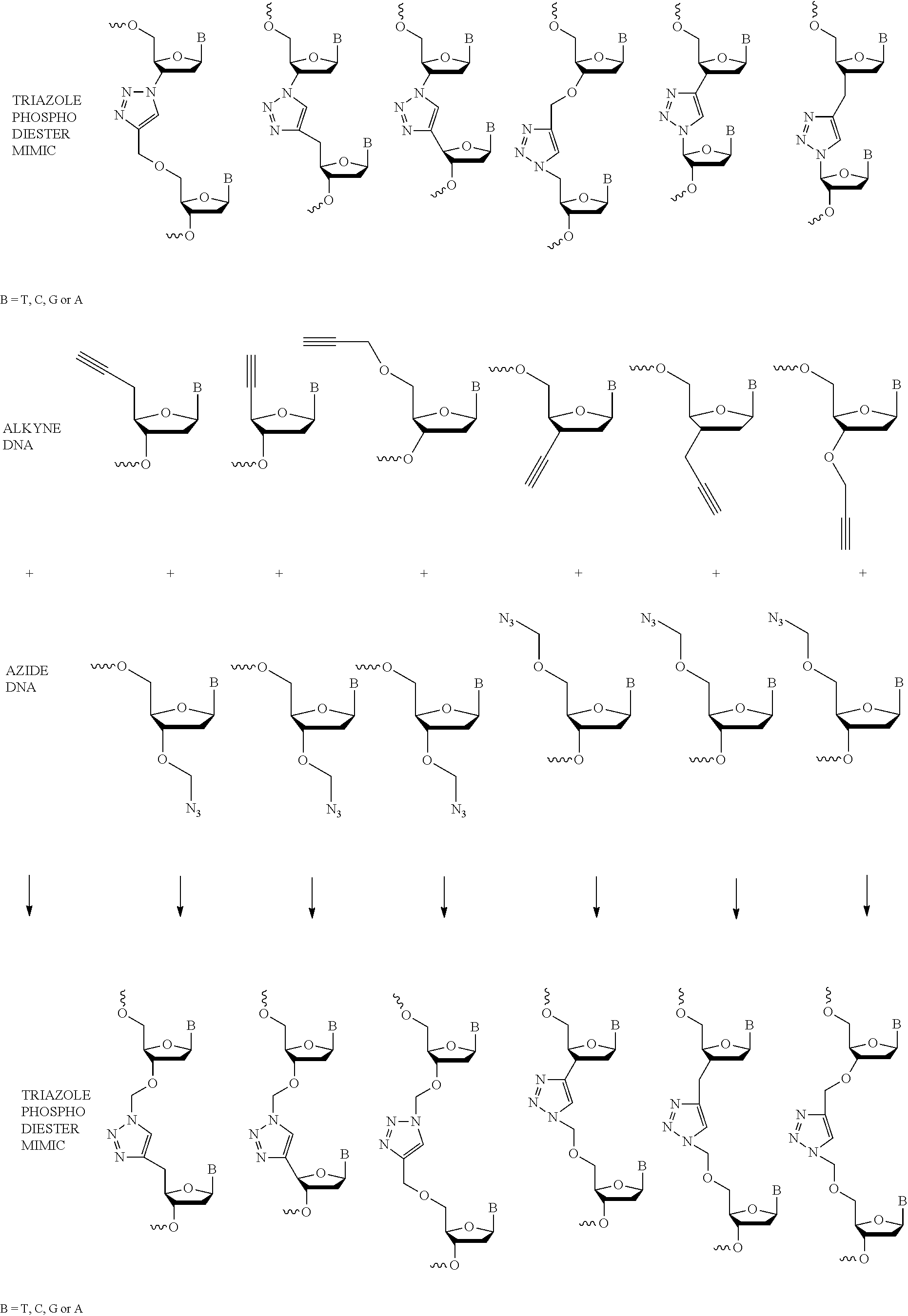

This method is advantageous because it employs a chemical synthesis reaction that is fast and can be performed on an industrial scale. In one embodiment the method of the present invention can be used to ligate together DNA or RNA molecules that have been produced chemically using phosphoramidite synthesis. In another embodiment the method of the present invention can be used to ligate together natural or enzymatically produced oligonucleotides to which alkynes or azides can be introduced to the 5′-end via 5′-alkyne or azide modified PCR primers and at the 3′-end by enzymes such as terminal transferase.

The oligonucleotides ligated by the method of the present invention may be made of DNA or RNA. In one embodiment two DNA oligonucleotides may be ligated together. In another embodiment two RNA oligonucleotides may be ligated together. In a further embodiment a DNA oligonucleotide may be ligated to an RNA oligonucleotide. In a further embodiment 2, 3, 4, 5, 6, 7, 8, 9, 10 or more than 10 oligonucleotides may be ligated together. In one embodiment this method may be used to ligate together oligonucleotides to form an oligonucleotide comprising more than 20, more than 40, more than 50, more than 80, more than 100, more than 200, more than 500, more than 800, more than 1000, more than 1500 or more than 2000 residues.

The method comprises reacting at least one alkyne group with at least one azide group. A suitable alkyne group may be chemically joined to the 3′ end of a DNA or an RNA oligonucleotide to provide an oligonucleotide that is useful in the present invention. A suitable azide group may be chemically joined to the 5′ end of a DNA or an RNA oligonucleotide to provide an oligonucleotide that is useful in the method of the present invention. Suitable alkyne and azide groups for use in the present invention are shown above. Alternatively the alkyne can be added to the 5′-end of an oligonucleotide and the azide to the 3′-end of an oligonucleotide. In one embodiment an alkyne group can be added to each end of an oligonucleotide. In another embodiment azide groups can be added to each end of an oligonucleotide. In a further embodiment an alkyne group can be added to one end of an oligonucleotide and an azide group can be added to the other end of the same oligonucleotide.

In one embodiment the reaction between an alkyne group and an azide group is an example of a type of reaction known as “click chemistry”. Ligating DNA and/or RNA molecules using a click chemistry reaction is advantageous because click chemistry reactions may be fast, modular, efficient, may not produce toxic waste products, can be done with water as a solvent and/or may be steriospecific.

In one embodiment, the present invention uses the CuAAC reaction for DNA and/or RNA ligation because of its very high speed, efficiency, orthogonality with functional groups present in nucleic acids, its compatibility with aqueous media, and the ability to switch on the reaction by adding Cu(I) after oligonucleotides have been annealed. In one embodiment, individual DNA or RNA oligonucleotides may be assembled by automated solid phase synthesis, purified by HPLC, then chemically ligated by click chemistry using the CuAAC reaction to produce much larger molecules. The CuAAC reaction may be catalyzed by Copper (I), which may be produced in the reaction mixture.

A Cu(I)-binding ligand may also be used to prevent Cu(I)-catalyzed oligonucleotide degradation.

In one embodiment the click reaction can be carried out on a solid-phase support, for example resin beads or a column comprising a suitable substrate or synthesis resin. The azide oligonucleotide can be left on a synthesis resin on an oligonucleotide synthesis column and the alkyne oligonucleotide can be added to the resin in the presence of aqueous Cu(I) so that the reaction occurs on solid-phase in a non-templated mode. This has the advantage that an excess of the alkyne oligonucleotide can be used to make the reaction very efficient. The excess unreacted alkyne oligonucleotide can be washed away leaving the ligated oligonucleotide (containing the triazole linkage) on the resin. This can then be cleaved from the resin and deprotected using standard procedures.

Alternatively the same procedure can be carried out with the alkyne oligonucleotide bound to the resin and the azide oligonucleotide in solution. An example of the reaction on a solid phase is shown in FIG. 10.

The reaction of at least one alkyne group with at least one azide group in the present invention may form at least one triazole phosphodiester mimic. The triazole phosphodiester mimic joins together two ribose or deoxyribose sugars or modified deoxyribose sugars in the backbone of DNA or RNA in place of a phosphate group. The triazole phosphodiester mimic may be comprised of a triazole ring and two linkers. One linker joins the triazole ring to the ribose or deoxyribose on one side of it and the other linker joins the triazole to the ribose or deoxyribose on the other side of it.

In one embodiment the at least one triazole phosphodiester mimic can be read through accurately by a DNA polymerase and/or an RNA polymerase. This means that a DNA and/or RNA polymerase correctly replicates or transcribes the sequence of the DNA and/or RNA at the site of the triazole phosphodiester mimic. For example, the DNA and/or RNA polymerase does not read the bases next to the triazole phosphodiester mimic incorrectly or skip a base near the site of the triazole phosphodiester mimic. This is advantageous because DNA and/or RNA molecules can be ligated together and the ligation product can be correctly read/copied by polymerases in vitro and/or in vivo.

In one embodiment a nucleic acid comprising at least one triazole phosphodiester mimic according to the present invention is active in vivo. For example the DNA or RNA sequence comprising at least one triazole phosphodiester mimic can direct the production of a functional polypeptide in living cells.

In one aspect the present invention relates to an oligonucleotide comprising one or more alkyne and/or azide groups that can be used in the method of the present invention. For example the present invention relates to an oligonucleotide linked to at least one alkyne group comprising a structure selected from the structures shown in FIG. 9. The present invention relates to an oligonucleotide linked to at least one azide group comprising a structure selected from the structures shown in FIG. 9.

In one embodiment the reaction to form a triazole phosphodiester mimic follows the reaction scheme below or an RNA equivalent thereof:

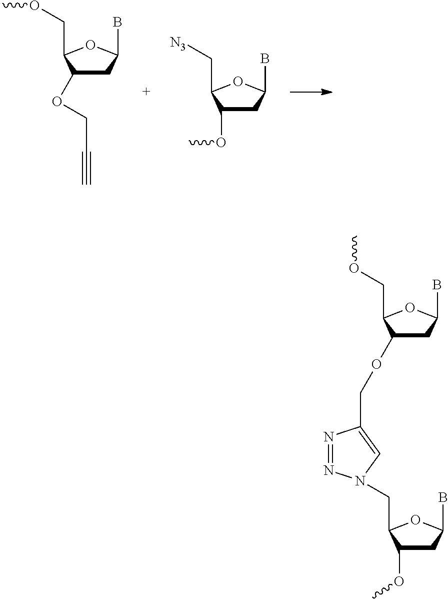

This reaction provides a triazole phosphodiester mimic that has an overall shape similar to that of a phosphodiester group. The similarity of the overall shape of this triazole phosphodiester mimic to a natural phosphodiester group can be seen in FIG. 7, which shows, at “B”, the phosphodiester group of canonical DNA and at “C” a superposition of the above triazole phosphodiester mimic with a phosphodiester group of canonical DNA. As can be seen, although the structures of these two linkers are different, the overall shape is similar. In addition, one or more of the nitrogen atoms of the triazole ring can form hydrogen bonds or electrostatic interactions with the polymerase enzyme in the same manner as the oxygen atoms of the natural phosphodiester group. Without being bound by theory, it is suggested that the similarity of shape of the triazole phosphodiester mimic means that the polymerase can pass across the triazole phosphodiester mimic without the normal activity of the polymerase being disrupted. This can be shown in FIG. 7 of the present application. FIG. 7C shows a triazole phosphodiester mimic of the present invention superimposed on a structure of a canonical phosphodiester group. As can be seen the shapes of the two groups are similar.

In one embodiment an alkyne group at the 3′ end of one oligonucleotide is reacted with an azide group which is at the 5′ end of a second oligonucleotide to form at least one triazole phosphodiester mimic. In one embodiment an alkyne group at the 3′ end of one single stranded oligonucleotide is reacted with an azide group which is at the 5′ end of a second single stranded oligonucleotide to form at least one triazole phosphodiester mimic in a single stranded oligonucleotide.

In one embodiment a single stranded oligonucleotide may be circularized by reacting an alkyne group at one end of the oligonucleotide with an azide group at the other end of the oligonucleotide to form a single stranded circular oligonucleotide comprising at least one triazole phosphodiester mimic.

In another embodiment double stranded hybridized oligonucleotides may be circularized by reacting an alkyne groups at one end of each strand with azide groups at the other end of each strand to form a circularized double stranded oligonucleotide, for example a catenane, comprising at least one triazole phosphodiester mimic. A catenane can also be formed by cyclizing one oligonucleotide and then using it as a template to cyclize a second oligonucleotide to make the double stranded catenane.

In another embodiment the method of the present invention may be used on oligonucleotides that form mixed single and double stranded nucleic acid structures such as hammerhead ribozymes, hairpin ribozymes or synthetic DNA and/or RNA constructs.

The ability to introduce one or more unnatural nucleotides into synthetically produced oligonucleotides and then to ligate them together using the methods of the present invention allows a wide range of non-natural oligonucleotide constructs to be made. These constructs can be made by the methods of the present invention without the use of enzymes and on a large scale by chemical synthesis.

In one embodiment the reaction of at least one alkyne group with at least one azide group may be carried out under non-templated conditions. This means that alkyne group attached to one oligonucleotide may be reacted with an azide group attached to the same or a different oligonucleotide in the absence of a template or a splint. The reaction proceeds in the presence of Cu(I) even if the two reaction oligonucleotides have no region of complementarity, i.e. a completely non-templated click reaction. In one embodiment an oligonucleotide with an alkyne group at one end and an azide group at the other will cyclize in the presence of Cu(I) even in the absence of a splint. The rate of a non-templated reaction may be increased by increasing the concentration of the oligonucleotides comprising one or more alkyne and/or azide groups.

The oligonucleotide or oligonucleotides comprising one or more alkyne and/or azide groups may self-assemble into the correct orientation for the ligation to take place. For example, one or more ends of the oligonucleotides may be complementary to the end of another oligonucleotide to which it can hybridize to orientate the oligonucleotides before the ligation reaction. These complementary ends may for example be “sticky ends” generated by cleavage of an oligonucleotide with a restriction enzyme.

In one embodiment the reaction of at least one alkyne group with at least one azide group may be carried out under templated conditions. In this embodiment a template oligonucleotide may be provided that will not take part in the ligation reaction or will not be ligated to an oligonucleotide that comprises one or more alkyne and/or an azide group. The oligonucleotide or oligonucleotides comprising one or more alkyne and/or azide groups may hybridize with the template. This is advantageous because it allows a two or more oligonucleotides to be assembled in the desired orientation to each other before ligating them together. In one embodiment the template may be an oligonucleotide for example a single stranded DNA or RNA oligonucleotide. In one embodiment the template may be a linear oligonucleotide, in another embodiment the template may be a circular oligonucleotide. In a further embodiment the template may be made using the method of the present invention and may comprise one or more triazole phosphodiester mimics as described in the present invention. The oligonucleotides comprising alkyne or azide groups can be hybridized to the template under suitable hybridization conditions that do not cause the alkyne and azide groups to react with one another. In one embodiment the reaction between the alkyne and azide groups proceeds very slowly in the absence of Copper(I) and the ligation reaction can be started once the oligonucleotides are annealed to the template in the right order by adding Copper (I) or by production of Copper (I) in the reaction solution at a suitable time.

In one embodiment the template may be a cyclic single-stranded oligonucleotide and a double-stranded helical oligonucleotide catenane is prepared.

In one embodiment the step of reacting at least one alkyne group with at least one azide group to form at least one triazole phosphodiester mimic may be repeated sequentially more than once, for example, more than twice, more than three times, more than four times, more than five times, more than six times or more than seven times, to form an oligonucleotide comprising more than one triazole phosphodiester mimic. The individual click reactions can be carried out sequentially.

In another embodiment several triazole linkages may be formed simultaneously if several alkyne/azide oligonucleotides are allowed to anneal to templates or to each other in the desired orientation and/or order and Cu(I) is added to the instigate the reaction. Four examples of schemes for making long oligonucleotides by ligating more than one oligonucleotide using a method of the present invention are shown in FIG. 11. Splints may be oligonucleotides that are complementary to the ends of the oligonucleotides that are to be joined together. The splints may anneal to the join between the oligonucleotides at the site of the alkyne azide reaction to make sure the oligonucleotides are in the correct orientation before they are joined together. After the alkyne azide reaction the splints may be removed. An alternative method of ensuring that the oligonucleotides comprising one or more alkyne and/or azide groups align in the correct orientation and order is to ensure that they are designed with complementary single-stranded ends that can anneal to each other as shown in FIG. 11, numbers 2 and 4.

An oligonucleotide in the present invention may be two or more, preferably 3 or more, preferably 5 or more, preferably 10 or more, preferably 20 or more, preferably 30 or more, preferably 40 or more, preferably 50 or more, preferably 100 or more, DNA nucleotides and/or RNA nucleotides and/or nucleotide analogues and/or labelled nucleotides linked by phosphodiester bonds. DNA and/or RNA analogues may be, for example 2′-O-methyl RNA, 2′-fluoro RNA and/or LNA.

In another embodiment at least one oligonucleotide is DNA and at least one oligonucleotide is RNA to form a DNA-RNA hybrid oligonucleotide.

The following triazole phosphodiester mimic structures are preferred in the present invention.
TRIAZOLE PHOSPHO DIESTER MIMICS B=T, C, G or A

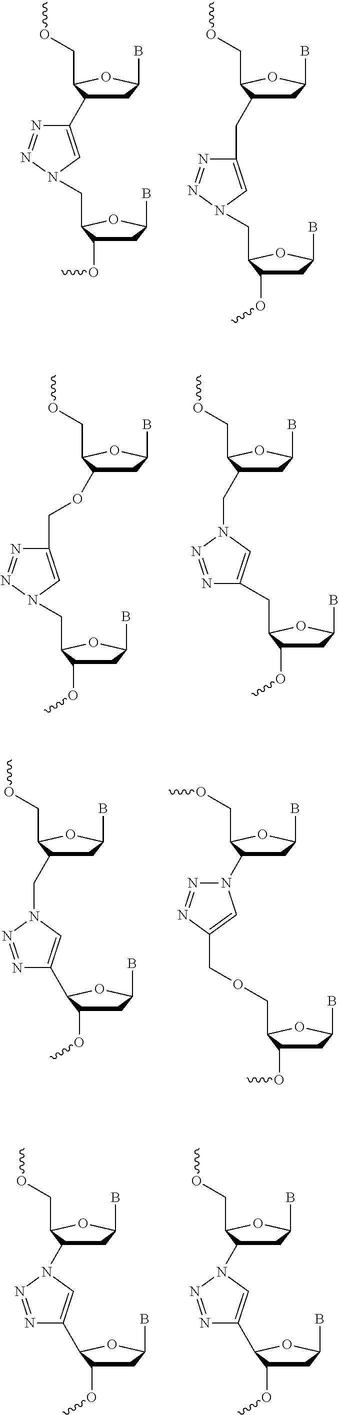

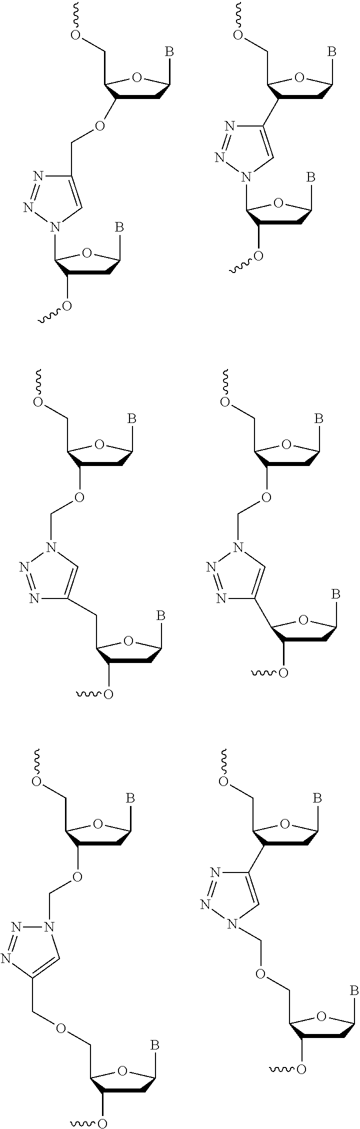

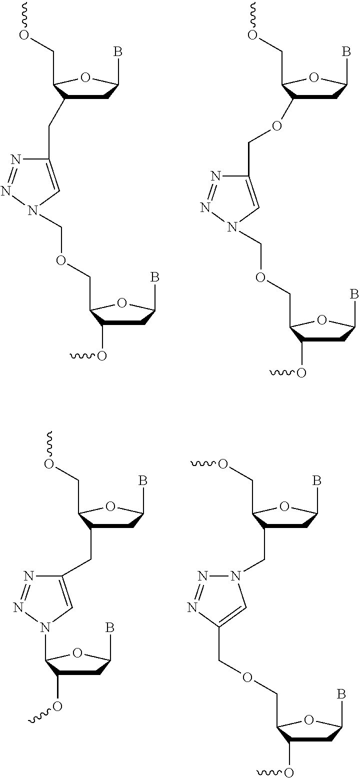

These structures are advantageous because they can be prepared using the CuAAC reaction by reacting an alkyne group at the end of one oligonucleotide with an azide group is at the end of a second oligonucleotide. This makes it fast and simple to use one of the above triazole phosphodiester mimic structures to link the backbones of two or more oligonucleotides together.

The triazole ring structure of each triazole phosphodiester mimic is linked to the ribose or deoxyribose structures by linkers. The oligonucleotides comprising one or more alkyne and/or azide groups for use in the reaction of the present invention can be designed in order to make any of the linkers shown above. In one embodiment a suitable linker may be chosen to suit the particular position in the oligonucleotide that the triazole phosphodiester mimic will occupy. For example linkers may be chosen to be on each side of the triazole ring in a triazole phosphodiester mimic so that the triazole phosphodiester mimic has the closest shape and/or size and/or charge distribution and/or hydrogen bonding characteristics and/or other physical properties possible to a natural phosphodiester bond. This makes it easier for a polymerase to correctly read through the triazole phosphodiester mimic and correctly replicate or transcribe the DNA. In another embodiment the linkers may be chosen to provide a desired shape to the oligonucleotide that is desirable for the design of an unnatural oligonucleotide construct.

In one embodiment the present invention relates to a method of selecting a triazole phosphodiester mimic comprising the steps of:

a) Designing an oligonucleotide construct;

b) Selecting a triazole phosphodiester mimic from those shown in FIG. 9 that has an appropriate shape and/or size and/or charge distribution and/or hydrogen bonding characteristics and/or other physical properties for the location in the oligonucleotide construct;

c) Designing oligonucleotides comprising alkyne and azide groups that can react to form the selected triazole phosphodiester mimic.

Optionally the method may also comprise the steps of:

d) Making the oligonucleotide construct designed in step a);

Testing the oligonucleotide construct to ensure that it contains a triazole phosphodiester mimic that is functional in vitro or in vivo.

The oligonucleotide construct may comprise at least two triazole phosphodiester mimics each having a structure selected from the constructs shown in FIG. 9 or an RNA equivalent thereof.

In one embodiment least one triazole phosphodiester mimic has the following structure or an RNA equivalent thereof:

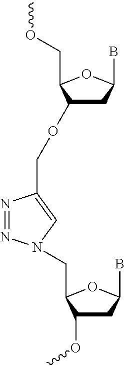

This is advantageous because this structure has a shape that closely resembles that of a canonical phosphodiester group. This triazole phosphodiester mimic is particularly suitable because it can be read through accurately by DNA and RNA polymerases. Oligonucleotides comprising this triazole phosphodiester mimic may be correctly copied by DNA polymerases and accurately transcribed by RNA polymerases in vitro and in vivo, for example they are able to direct the expression of polypeptides in E. coli cells and hammerhead ribozymes have been shown to be active with this triazole phosphodiester mimic at the active site.

The method of the present invention and the triazole phosphodiester mimics of the present invention provide a number of advantages. These include:

The triazole phosphodiester mimics can be read through by a DNA and RNA polymerases. Oligonucleotides linked to an alkyne group and or an azide group for use in the method of the present invention may be easy to prepare.

The oligonucleotides comprising one or more azide and/or alkyne groups can be made on a large scale by chemical synthesis of the oligonucleotides and chemically joining an alkyne or an azide group to the oligonucleotide. The oligonucleotides can be synthesized to include unnatural nucleotides. Labels, such as fluorescent labels and epigenetic structures such as methylated or hydroxymethylated nucleobases because there is no requirement for enzymatic synthesis.

The ligation of oligonucleotides can be done on a large scale, for example at least one gram, preferably at least 10 grams, preferably at least 50 grams, preferably at least 100 grams, preferably at least 500 grams, preferably at least 1 kilogram, preferably at least 2 kilograms of product may be produced in a single reaction.

In one embodiment there is no requirement to purify the ligation products to remove enzymes.

The chemical ligation reaction can be initiated at any time by the addition of Cu(I) and will not occur at a measurable rate in the absence of Cu(I). Therefore, the participating oligonucleotides can be allowed to slowly anneal to each other or to a template and produce the correct construct before the reaction is initiated, thus avoiding the formation of incorrect products.

### BRIEF DESCRIPTION OF THE DRAWINGS

There now follows by way of example only a detailed description of the present invention with reference to the accompanying drawings, in which;

FIG. 1 shows DNA linkage structures: 1a. Canonical DNA, 1b. Previous triazole DNA analogue described in El-Sagheer et al., (2009), J Am Chem Soc 131, 3958-3964. 1c. Biocompatible triazole analogue, 1d. Click ligation to produce triazole DNA mimic 1c. 1e. Polymerases read through 1b using only one of the two thymines as a template base, i.e. TₜT→T. (t=triazole). 1f. PCR copies the base sequence around the unnatural linkage 1c correctly.

FIG. 2 shows a scheme for the synthesis of alkyne/azide oligonucleotides for use in click ligation and cyclization: 2a. Assembly of 3′-alkyne oligonucleotide. 2b. Conversion to 5′-azide. Oligonucleotides can be made with 5′-azide, 3′-alkyne or both. A dinucleotide is shown for clarity but the reactions have been carried out on oligonucleotides up to 100-mer in length. 2c. 3′-Propargyl dT introduced as final addition in reverse phosphoramidite assembly of DNA.

FIG. 3 shows cyclization and RCA of 5′-azide-3′-alkyne 100-mer: 3a. Reversed-phase HPLC (UV abs at 260 nm) and 3b. mass spectrum (ES⁻) of cyclic 100-mer ODN-31 (SEQ. ID. No. 31), required; 31.423 kDa, found 31.422 kDa. 3c. Schematic of RCA reaction. 3d. RCA product from cyclic 100-mers using phi29 DNA polymerase. Lane 1; 50 bp DNA ladder, lanes 2 and 3; RCA of cyclic triazole ODN-31 (SEQ. ID. No. 31) and cyclic normal ODN-49 (SEQ. ID. No. 49) respectively.

FIG. 4 shows PCR amplification of 210-mer and 300-mer click-ligated triazole DNA templates: 4a. Schematic representation of click ligation of three oligonucleotides. 4b. Click ligation reaction: Lane 1; crude reaction mixture to synthesize 210-mer template from three 70-mers, lane 2; starting oligonucleotide ODN-16 (SEQ. ID. No. 16) (8% polyacrylamide gel). 4c. PCR using 210-mer triazole template. Lane 1; 25 bp DNA ladder, lane 2; control PCR without click-ligated template, lane 3; PCR using 210-mer triazole template. 4d. PCR using 300-mer triazole template. Lane 1; 25 bp DNA ladder, lanes 2, 3; PCR using short primers ODN-26 (SEQ. ID. No. 26) and ODN-27 (SEQ. ID. No. 27), Lanes 4, 5; PCR using long primers ODN-28 (SEQ. ID. No. 28) and ODN-29 (SEQ. ID. No. 29). (2% agarose gels with ethidium staining). 4e. Sequencing data from 300-mer triazole amplicon showing that the base sequence of the template was replicated faithfully at the two ligation sites.

FIG. 5 shows assembly of a T7-Luciferase control plasmid containing click-DNA within its BLA gene. A region corresponding to the central part of BLA was PCR-amplified using oligonucleotide primers (ODN-39 (SEQ. ID. No. 39), ODN-41 (SEQ. ID. No. 41)) containing triazole linkage 1c. The PCR product was ligated into the digested plasmid to give an intact construct containing triazole linkages on each strand of its BLA gene.

FIG. 6 shows biocompatability of click DNA in E. coli: 6a. The plate on the left is the negative control (no insert), the middle plate contains transformants of plasmids with the triazole DNA insert in its BLA gene (127 colonies), and the plate on the right is the native plasmid (129 colonies). 21 replicates of each plate were performed. 6b, c. Sequencing of the BLA gene from colonies in the triazole DNA plates. In c, the ^{Me}CₜC is contained on the complementary strand, therefore appearing as GG. 6d. Comparison of colony growth in the control (C), native (N) and triazole (T) plates. Triazole plates contained 96.5% of the colonies in the native plates (S.D.=1.6%) whereas the negative control was 1.1% (S.D.=1.0%).

FIG. 7 shows Taq polymerase primer-template dNTP closed complex: 7a. Schematic of interactions between the phosphodiester linkages of the DNA template and amino acids of the enzyme. Only template strand is shown. mc=main chain. 7b. Canonical DNA; the majority of the interactions with the polymerase involve the branched phosphate oxygen atoms, few if any involve bridging oxygen atoms. 7c. Overlay of canonical DNA and triazole linkage 1c. The N2 and N3 atoms of triazoles are good hydrogen bond acceptors and in principle they could substitute for the phosphate oxygen atoms. 7d. Triazole linkage 1b showing the trans-configuration of the amide, with N2 and N3 of the triazole facing into the helix. Linkage 1b is significantly longer than 1a and 1c.

FIG. 8 shows a diagram of a DNA catanane. This is a double stranded closed circle of DNA which is not supercoiled.

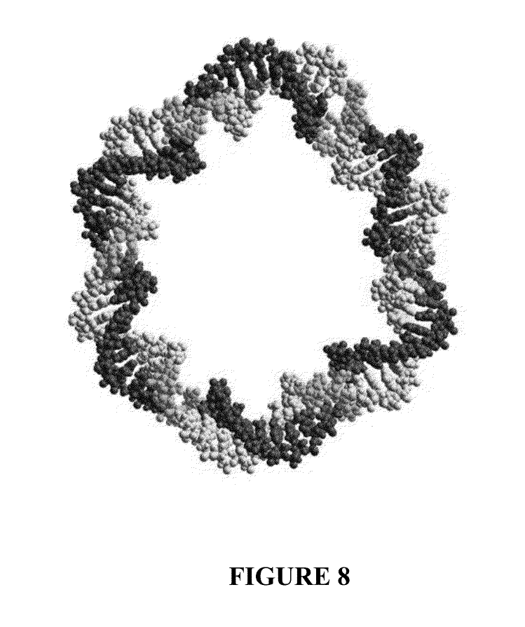

FIG. 9 shows examples of alkyne and azide pairs and the triazole phosphodiester mimics that they can form when reacted with each other. In each case only the deoxyribose that is linked to the alkyne or azide is shown but this deoxyribose may form part of an oligonucleotide. These examples show alkyne and azide pairs linked to deoxyribose but, it is also invisaged that these alkyne and azide groups can be linked to ribose groups of RNA in the same way. In each example B=a base, for example one of the five bases that occur in DNA and RNA (A, G, C, T and U) or a modified base. In the bottom line of each section triazole phosphodiester mimics that can be formed from the alkyne and azide pairs are shown.

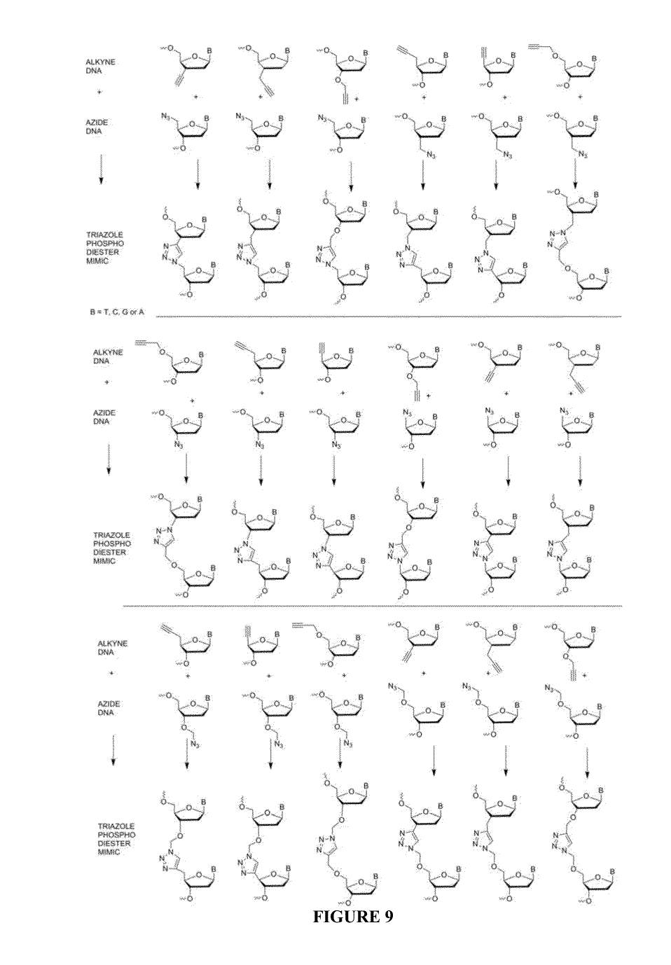

FIG. 10 shows a diagram of the reaction of an azide attached to an oligonucleotide and an alkyne attached to an oligonucleotide where the reaction takes place on a solid phase, in this case a synthesis resin. The azide oligonucleotide can be left on the synthesis resin on the oligonucleotide synthesis column and the alkyne oligonucleotide can be added to the resin in the presence of aqueous Cu(I) so that the reaction occurs on solid-phase in a non-templated mode. This has the advantage that an excess of the alkyne oligonucleotide can be used to make the reaction very efficient. The excess unreacted alkyne oligonucleotide can be washed away leaving the ligated oligonucleotide (containing the triazole linkage) on the resin. This can then be cleaved from the resin and deprotected using standard procedures. Alternatively the same procedure can be carried out with the alkyne oligonucleotide bound to the resin and the azide oligonucleotide in solution.

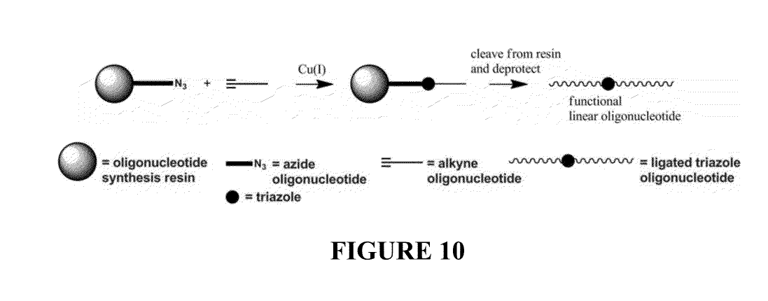

FIG. 11 shows four examples of schemes for making long oligonucleotides from one or more short oligonucleotides by ligating them together using the method of the present invention. In scheme one splints are used. These are short oligonucleotides that are complementary to two ends of oligonucleotides that will be ligated together. The splints ensure that the oligonucleotides are ligated together in the right order and orientation. In scheme 2 oligonucleotides with alkyne and/or azide groups attached are designed to have complementary overlapping ends, for example sticky ends. The overlapping complementary ends ensure that the oligonucleotides ligate to each other in the correct order and orientation. In scheme 3 complementary splints are used to construct a circular single stranded oligonucleotide. In scheme 4 oligonucleotides with overlapping complementary ends are used to construct a circular double stranded oligonucleotide. Several triazole linkages can be formed simultaneously if several alkyne/azide oligonucleotides are allowed to anneal and Cu(I) is added to the instigate the reaction. Alternatively the individual click reaction can be carried out sequentially. An oligonucleotide may have one alkyne or one azide group attached to one end or may have an alkyne group at each end, an azide group at each end or an alkyne group at one end and an azide group at the other end.

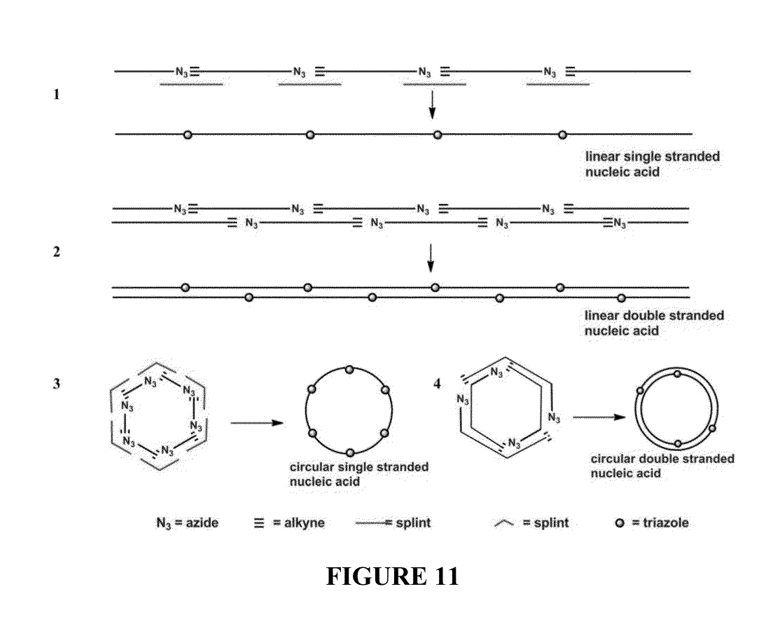

### DETAILED DESCRIPTION

A triazole mimic of a DNA phosphodiester linkage has been produced by templated chemical ligation of oligonucleotides functionalized with 5′-azide and 3′-alkyne. The individual azide and alkyne oligonucleotides were synthesized by standard phosphoramidite methods and assembled using a straightforward ligation procedure. This highly efficient chemical equivalent of enzymatic DNA ligation has been used to assemble a 300-mer from three 100-mer oligonucleotides, demonstrating the total chemical synthesis of very long oligonucleotides. The base sequences of the DNA strands containing this artificial linkage were copied during PCR with high fidelity, and a gene containing the triazole linker was functional in E. coli.

Solid-phase DNA synthesis is an advanced technology that has led to pioneering discoveries in biology and nanotechnology. Although automated solid-phase phosphoramidite synthesis is highly efficient, the accumulation of modifications (mutations) and failure sequences caused by side-reactions and imperfect coupling imposes a practical limit of around 150 bases on the length of oligonucleotides that can be made. Consequently very long synthetic oligonucleotides are not suitable for use in biological applications that require sequence fidelity, so combinations of shorter sequences are normally used in PCR-mediated gene assembly. This enzymatic method of DNA synthesis has the intrinsic limitation that site-specific chemical modifications can only be introduced in the primer regions of the resulting constructs. Certain unnatural analogues can be inserted throughout the PCR amplicon via modified dNTPs, but this process is essentially uncontrolled and does not allow combinations of different modifications to be incorporated at specific loci. Therefore, for biological studies, important epigenetic and mutagenic bases such as 5-methyl dC, 5-hydroxymethyl dC and 8-oxo dG are normally put into short oligonucleotides and subsequently inserted into larger DNA strands by enzymatic ligation. Templated enzymatic ligation of oligonucleotides can be used to produce large DNA fragments, but this is best carried out on a small scale. In addition, some modified bases are not tolerated by ligase enzymes. Enzymatic methods of gene synthesis are extremely important in biology, but a purely chemical method for the assembly of large DNA molecules would be an interesting and valuable addition to current tools, with the advantages of scalability, flexibility and orthogonality.

It has proved challenging to achieve clean and efficient chemical ligation of canonical DNA, although significant progress has been made using cyanogen bromide as a coupling agent. An interesting alternative approach is to design a chemical linkage that mimics the natural phosphodiester group, and which can be formed in high yield in aqueous media from functional groups that are orthogonal to those naturally present in DNA.

Three key requirements of the strategy of the present invention are the use of functional groups that are highly stable in aqueous media, the ability to selectively initiate the ligation reaction only when participating oligonucleotides have been hybridized to complementary splints (to arrange the DNA strands in the desired order by templated pre-assembly), and the creation of a very stable backbone linkage. The present invention relates to a high-yielding DNA ligation method (click ligation) based on the CuAAC reaction. Click chemistry has previously been used in the nucleic acids field but previous DNA triazole linkages were not accurately read through by PCR. Amplification of the resulting modified DNA template caused the loss of one nucleotide at the site of click ligation. The consistently observed deletion mutation in the resulting PCR products indicated that this previous artificial DNA linkage was not an adequate mimic of a phosphodiester group and it does not behave like its natural counterpart in vivo.

The present invention relates to the synthesis and properties of newly designed triazole phosphodiester mimics using oligonucleotides comprising alkyne or azide groups that can be readily prepared by standard phosphoramidite methods, and that are functional both in vitro, as substrates for DNA and RNA polymerases. It is the first example of a biocompatible artificial DNA linkage that can be formed efficiently by chemical ligation.

### Synthesis and Assembly of Azide/Alkyne Oligonucleotides

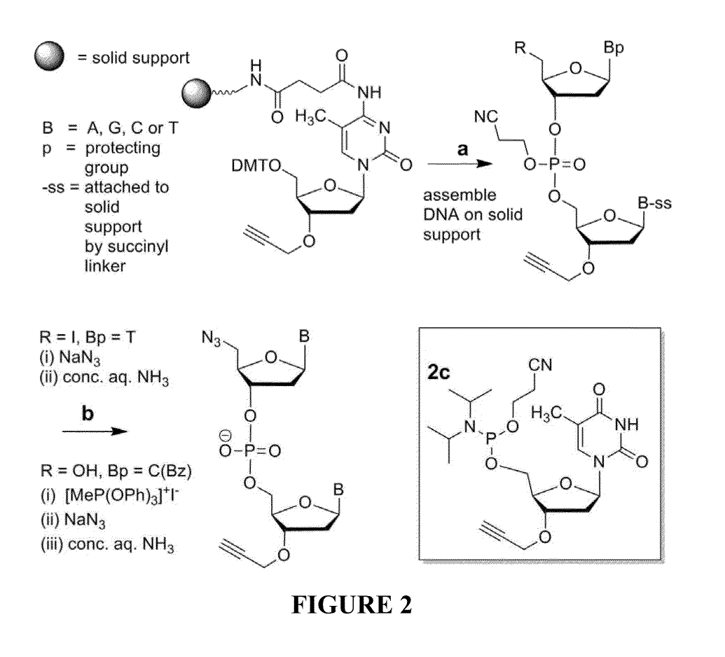

The triazole phosphodiester mimic of the present invention has the considerable advantage of being constructed from oligonucleotides made entirely by the phosphoramidite method, one bearing a 5′-azide functional group and the other a 3′-alkyne. The functionalized resin required for the solid-phase synthesis of oligonucleotides terminating with 3′-propargyl ^{Me}dC (cytosine equivalent, (FIG. 2) was prepared from thymidine as previously described in El-Sagheer A H & Brown T (2010), “New strategy for the synthesis of chemically modified RNA constructs exemplified by hairpin and hammerhead ribozymes.” Proc. Natl. Acad. Sci. U.S.A. 107(35):15329-15334. A polystyrene support was used to achieve high coupling yields and produce 100-mer oligonucleotides of the purity required for efficient click ligation. DNA strands containing 3′-propargyl dT were made from reverse phosphoramidites which required the synthesis of monomer 2c. The 5′-azide group was introduced in a 2-stage process (FIG. 2); the 5′-OH group of a normal support-bound oligonucleotide was first converted to 5′-iodo by reaction with methyltriphenoxyphosphonium iodide (for oligonucleotides with 5′-dT this was simplified by direct incorporation of 5′-iodo thymidine phosphoramidite), then the resultant 5′-iodo oligonucleotides were reacted with sodium azide to complete the transformation. Oligonucleotides functionalized with both 3′-alkyne and 5′-azide were made by performing oligonucleotide synthesis on 3′-propargyl ^{Me}dC resin then converting the 5′-terminus to azide as described above. In this study the bases on either side of the triazole linkage are thymine and cytosine (or 5-methylcytosine). This is an adequate combination for the synthesis of any large DNA strand by click ligation, but it may be possible to use the same methodology for other combinations of nucleosides.

### Amplification of Click-DNA by Thermostable Polymerases

To investigate the compatibility of linkage 1c with thermostable polymerases, three 81-mer DNA templates were synthesized, each containing a single triazole linkage. PCR of these constructs produced amplicons that were faithful copies of the original sequence, with the TₜT, TₜC, ^{Me}CₜT and ^{Me}CₜC linkages being read through accurately (ₜ=triazole 1c). It is possible however, that PCR amplification of the chemically modified DNA might appear to be efficient even if read-through of the artificial linkage is a rare event. The ability of DNA polymerases to replicate through the triazole linkage was therefore evaluated more rigorously by linear copying of an 81-mer (ODN-08 (SEQ. ID. No. 08), Table S1, infra) using Large Klenow fragment. The reaction was efficient and the full length product was obtained in less than 5 minutes.

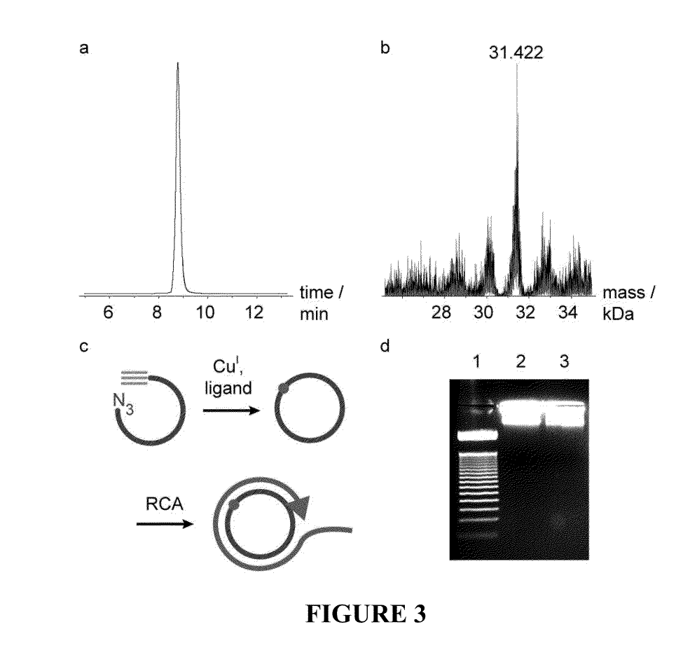

Application of click DNA ligation to the synthesis of large linear DNA constructs requires oligonucleotides that are functionalized at both termini. Simultaneous ligation of three 11-mer oligonucleotides in the presence of a complementary 41-mer splint was evaluated. The click ligation reaction was clean (SI Appendix Figure S4) and the product was characterized by ESI mass spectrometry (calc. 10064, found 10064). The integrity of the terminal alkyne and azide is essential for efficient click ligation, so it was important to show that the large numbers of repeated steps employed in the synthesis of long oligonucleotides do not destroy these functional groups. This was confirmed by successfully cyclising a 100-mer with 5′-azide and 3′-alkyne functionalities. The reaction proceeded smoothly in the absence of a complementary template oligonucleotide and the product was characterized by gel-electrophoresis and mass spectrometry (FIGS. 3a and b). Under similar conditions enzymatic cyclization failed, whereas in templated mode both the chemical and enzymatic cyclization reactions were successful.

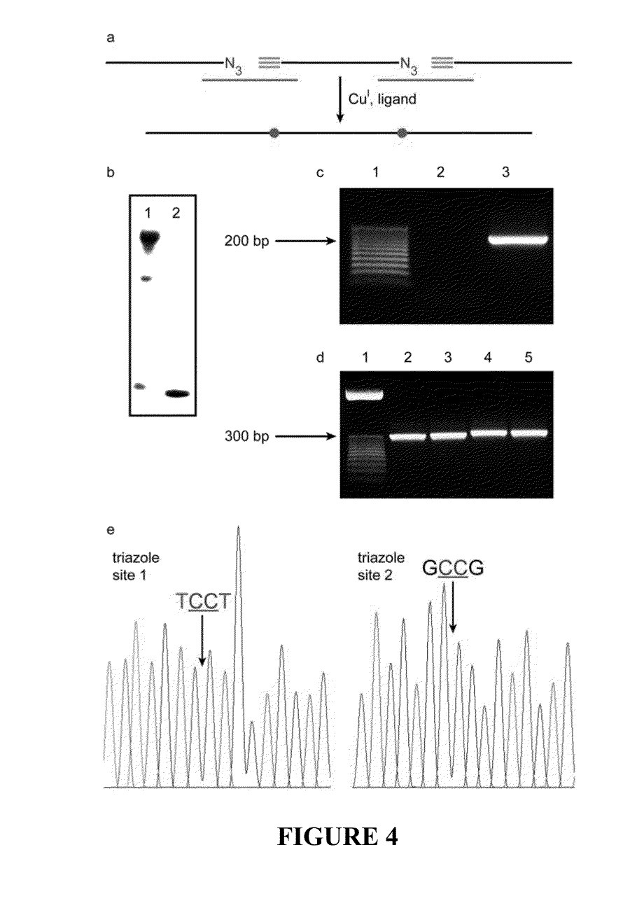

To demonstrate the utility of click ligation for the assembly of large DNA molecules, a 210-mer PCR template was assembled from three 70-mers, as well as a 300-mer from three 100-mer oligonucleotides. The oligonucleotides were designed to have an even distribution of A, G, C and T bases and to be devoid of secondary structure. The ligation products were purified by gel-electrophoresis and used as templates in PCR, after which the amplified regions were cloned, sequenced and found to be correct (FIG. 4). Thermostable polymerases with or without proofreading activity (Pfu and GoTaq respectively) read through the sequence around the click linkers to give the expected amplicons. In this study a total of four different base stacking steps (YₜY, all possible combinations of pyrimidines) on either side of the triazole were examined in several different tetramer sequences (Table S1), and in all cases (134 clones) the bases encompassing the triazole linkages were replicated correctly. In addition to PCR, rolling circle amplification (RCA) of a cyclic 100-mer containing a triazole linkage were carried out using the highly processive phi29 polymerase. The cyclic template was produced in an intramolecular click ligation reaction of a 5′-azide-3′-alkyne oligonucleotide (ODN-30 (SEQ. ID. No. 30), Table S1, infra). An essentially identical profile of phi29 RCA products was obtained from both normal and triazole cyclic templates (FIG. 3d), and long RCA products using GoTaq polymerase under standard PCR cycling conditions, by repeated read-through of the triazole linkage in a short timescale. The amplified RCA product was probed with a fluorescent HyBeacon to confirm that it was a true copy of the original template rather than a non-specific amplification product.

Biocompatibility of the Click Linker in E. coli

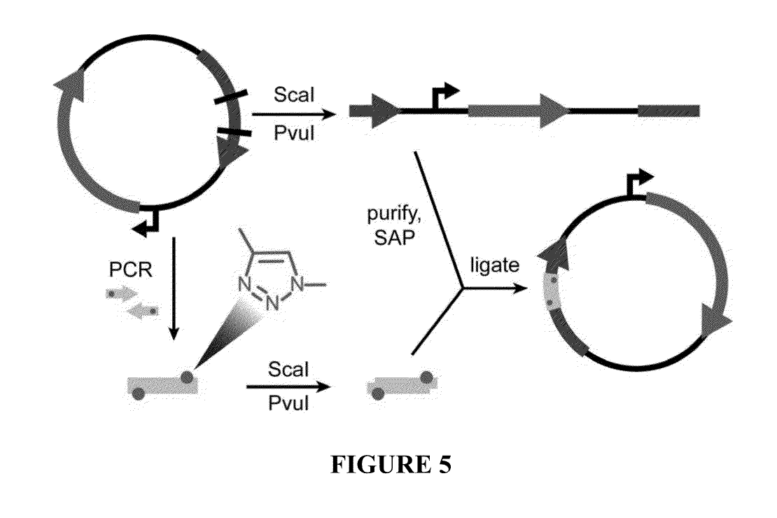

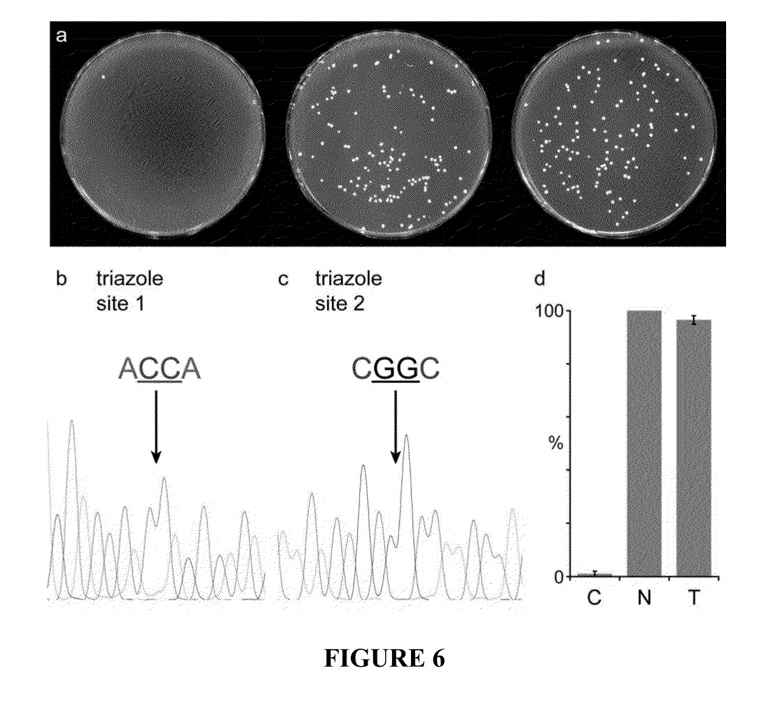

Following the successful in vitro experiments the biocompatibility of the modified DNA was investigated in vivo within the cellular machinery of E. coli (FIG. 5) by constructing a plasmid containing a triazole linkage in each strand of its antibiotic marker gene. The triazole linkages were introduced via modified PCR primers that amplify a portion of the TEM-1 β-lactamase (BLA) gene between the ScaI and PvuI restriction sites. PCR with these primers yielded a product matching the middle section of BLA, containing ^{Me}CₜC near the 3′ terminus of each strand. Electrophoresis of the amplicon showed it to be of the expected size and identical in length to that from the control PCR carried out with unmodified primers. The products of both PCR reactions (using unmodified and modified primers) as well as a plasmid containing the BLA gene (T7-Luciferase control, Promega Inc.) were digested with ScaI and PvuI restriction endonucleases. The digested plasmid (now lacking the region between ScaI and PvuI in its BLA gene) was gel-purified to remove the insert and undigested/singly digested plasmid, and treated with shrimp alkaline phosphatase to remove the phosphate monoesters from the 5′-termini to prevent self-ligation. The digested PCR products were then ligated into the linearized plasmid backbone via the matching ScaI and PvuI sites using T4 DNA ligase. A control ligation reaction was also set-up containing water in place of the insert to measure the level of ampicillin resistance arising from the presence of partially digested or undigested backbone. The resulting ligation mixtures were transformed into E. coli (NEB 5α) and grown on LB-agar plates containing 100 μg/mL of ampicillin (21 plates of each type). After overnight incubation at 37° C. the number of colonies from the triazole plasmids was 96.5% of the native, whereas the negative control was only 1.6% (FIG. 6). Plasmid was isolated and sequenced the BLA gene from 50 of the surviving colonies on both the positive control and the triazole DNA plates. In all cases the base sequence at the ^{Me}CₜC linkage was copied correctly.

The survival and growth of colonies containing a triazole-modified antibiotic marker gene suggests that the sequence around the triazole linkage is amplified correctly by the E. coli polymerases. However, viability might also be maintained if the region surrounding the triazole modification was excised by the cellular DNA repair machinery via nucleotide excision repair (NER) and replaced by a phosphodiester linkage. This possibility was investigated using a UvrB-deficient strain of E. coli (JW0762-2). UvrB is a central component of NER, interacting with UvrA, UvrC, UvrD, DNA polymerase I and DNA during excision-repair. If the biocompatibility of the click DNA linker was a consequence of NER, repair-deficient colonies would not survive on selective media when transformed with the triazole plasmid. Transformation of the repair-deficient strain of E. coli with the triazole plasmid gave 93% of the number transformed with the native plasmid, and sequencing the BLA gene from 21 of the colonies revealed that the region around the triazole linkage was copied correctly in all cases. This strongly supports the hypothesis that NER does not make a significant contribution to the biocompatibility of the triazole linkages.

### Rationale for Biocompatibility of Triazole Linkage in DNA

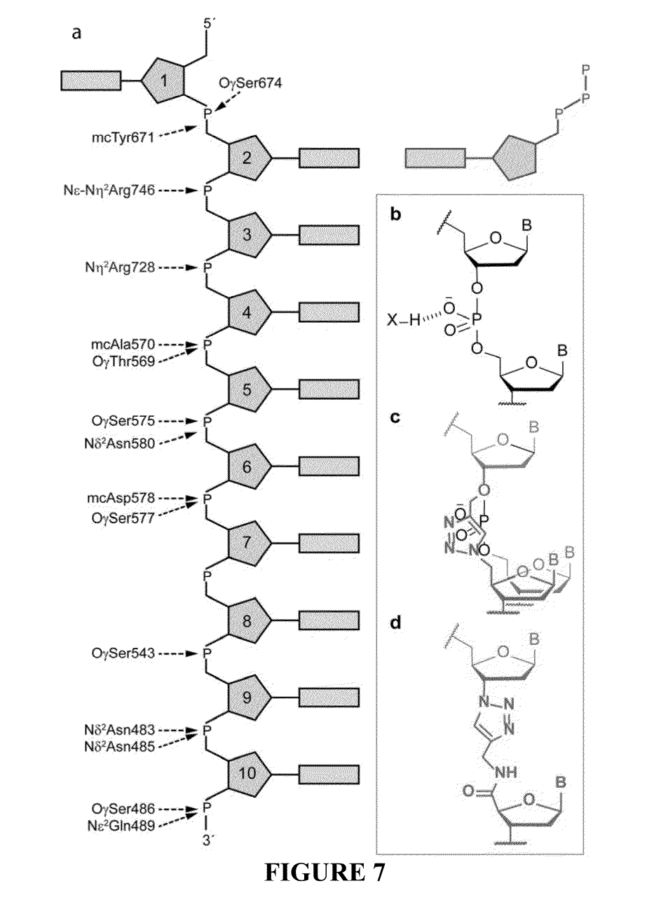

The ability of DNA polymerases to accurately synthesize a complementary copy of an artificial DNA linkage that bears limited structural resemblance to a natural phosphodiester may seem surprising. However, without wishing to be bound by theory, the X-ray structure of the Klenow fragment of Taq polymerase (Klentaq-1) with double stranded DNA at its active site provides some insight into the underlying mechanism of this phenomenon. In this structure there are several polar interactions between the enzyme and the phosphodiester groups of the DNA template strand that are consistent with hydrogen bonding (FIG. 7a). As the polymerase passes through the chemically modified template-primer complex, only one of the ten template nucleotides bound to the enzyme at any given time can encompass a triazole. Hence a maximum of only two interactions can be disrupted by the modification. In addition, some enzyme binding at the triazole site could still occur, as the triazole moiety has a large dipole moment and well-characterized hydrogen bond acceptor capacity. The requirement for dynamic and non-specific binding between DNA and the enzyme might also explain why the presence of triazole linkage 1c does not compromise fidelity during PCR amplification. A similar picture of enzyme template binding emerges from the structure of DNA bound to Taq polymerase, a version of the enzyme that has 3′-exonuclease activity.

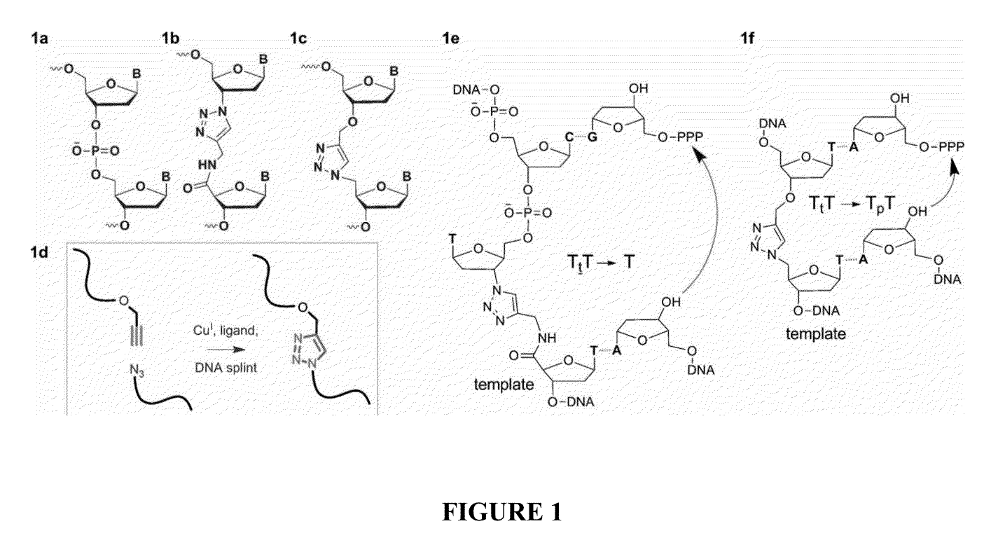

It was postulated that linkage 1c with its 3′-oxygen, 5′-methylene and greater conformational flexibility is a closer analogue of a natural phosphodiester than 1b. In contrast to 1c, it is apparent that triazole 1b alters the characteristics of the DNA sufficiently to prevent faithful replication. The thymine base on the 5′-side of the triazole may not be presented at the polymerase domain in a suitable orientation to base pair with the incoming dATP, so the only option is for replication to continue from the next available template base (FIG. 1e). In addition, linkage 1b is by no means an obvious phosphodiester surrogate in terms of H-bonding acceptor capacity, so its binding to the polymerase may be compromised. The normally favored trans-configuration at the amide bond, and the extended length of this linkage, may not allow the N2 and N3 atoms of the triazole to substitute for phosphodiester oxygen atoms (FIG. 7d).

Regardless of the detailed mechanisms, the results indicate that the artificial DNA linker is remarkably biocompatible, and investigations are underway to solve the high-resolution structure of a DNA duplex containing this triazole linkage and determine its effects on DNA conformation and dynamics.

### Materials and Methods

All oligonucleotide sequences are given in Table S1 below:

TABLE S1
Oligonucleotides used

| Code | Oligonucleotide sequences (5′-3′) |
|---|---|
| ODN-01 | ^{z}TACCACACAATCTCACACTCTGGAATTCACACTGACA |
| (SEQ. ID. | ATACTGCCGACACACATAACC |
| No. 01) | |
| ODN-02 | ^{z}CAGCACACAATCTCACACTCTGGAATTCACACTGAC |
| (SEQ. ID. | AATACTGCCGACACACATAACC |
| No. 02) | |
| ODN-03 | GCATTCGAGCAACGTAAGATCG^{Me}C^{k} |
| (SEQ. ID. | |
| No. 03) | |
| ODN-04 | gcattcgagcaacgtaagatcct^{k} |
| (SEQ. ID. | |
| No. 04) | |
| ODN-05 | gcattcgagcaacgtaagatcgt^{k} |
| (SEQ. ID. | |
| No. 05) | |
| ODN-06 | GCATTCGAGCAACGTAAGATCCTₜTACCACACAATCTC |
| (SEQ. ID. | ACACTCTGGAATTCACACTGACAATACTGCCGACACA |
| No. 06) | CATAACC |
| 81-mer | |
| ODN-07 | GCATTCGAGCAACGTAAGATCGTₜCAGCACACAATCT |
| (SEQ. ID. | CACACTCTGGAATTCACACTGACAATACTGCCGACAC |
| No. 07) | ACATAACC |
| 81-mer | |
| ODN-08 | GCATTCGAGCAACGTAAGATCG^{Me}CₜCAGCACACAATC |
| (SEQ. ID. | TCACACTCTGGAATTCACACTGACAATACTGCCGACA |
| No. 08) | CACATAACC |
| 81-mer | |
| ODN-09 | GCATTCATGT^{Me}C^{k} |
| (SEQ. ID. | |
| No. 09) | |
| ODN-10 | ^{z}CTGGTCCGTG^{Me}C^{k} |
| (SEQ. ID. | |
| No. 10) | |
| ODN-11 | ^{z}CGCGTCTAACC |
| (SEQ. ID. | |
| No. 11) | |
| ODN-12 | GCATTCATGT^{Me}CₜCTGGTCCGTG^{Me}CₜCGCGTCTAACC |
| (SEQ. ID. | |
| No. 12) | |
| 33-mer | |
| ODN-13 | 5TTTTGGTTAGACGCGGCACGGACCAGGACATGAATG |
| (SEQ. ID. | CTTTT |
| No. 13) | |
| ODN-14 | ^{z}TCGGTCGTCGAATTCTAGTAGATGTCTACATGTACAA |
| (SEQ ID. | CATACGCGCAGACGTATAGACTATCGCTCGTG^{Me}C^{k} |
| No. 14) | |
| ODN-14a | CGGTCGTCGAATTCTAGTAGATGTCTACATGTACAAC |
| (SEQ. ID. | ATACGCGCAGACGTATAGACTATCGCTCGTG^{Me}CₜT |
| No. 14a) | Same sequence as linear ODN-14 |
| Cyclic | (SEQ. ID. No. 14) with ^{Me}CₜT |
| | in the cyclic construct |
| ODN-15 | GCATTCGAGCAACGTAAGATCCTGAACTGGCATGACG |
| (SEQ. ID. | GTATGACACTGGCATGCTGTGACAGCATATGT^{Me}C^{k} |
| No. 15) | |
| ODN-16 | ^{z}TGCGTCGTCTGAGCAGTCTGATCGTGTCTGAGTACGG |
| (SEQ. ID. | CATTACCAGACAATACTGCCGACACACATAACC |
| No. 16) | |
| ODN-17 | TACTAGAATTCGACGACCGAGACATATGCTCTCACAG |
| (SEQ. ID. | CAT |
| No. 17) | |
| splint | |
| ODN-18 | CAGACTGCTCAGACGACGCAGCACGAGCGATAGTCTA |
| (SEQ. ID. | TAC |
| No. 18) | |
| splint | |
| ODN-19 | GCATTCGAGCAACGTAAGATCCTGAACTGGCATGACG |
| (SEQ. ID. | GTATGACACTGGCATGCTGTGAGAGCATATGT^{Me}CₜTC |
| No. 19) | GGTCGTCGAATTCTAGTAGATGTCTACATGTACAACA |
| 210-mer | TACGCGCAGACGTATAGACTATCGCTCGTG^{Me}CₜTGCG |
| | TCGTCTGAGCAGTCTGATCGTGTCTGAGTACGGCATT |
| | ACCAGACAATACTGCCGACACACATAACC |
| ODN-20 | ^{z}CTGGTCGTCGAATTCTAGTAGATGTCTACATGTAACAG |
| (SEQ. ID. | ATGTCGATACGCCAGTACGCGCTAGGATCACATACGC |
| No. 20) | GCAGACGTATAGACTATCGCTCGTG^{Me}C^{k} |
| ODN-21 | ^{z}CGCGTCGTCTGAGCAGTCTGATCGTGTCTGAGTACGC |
| (SEQ. ID. | ATGATCTGGATGTGTGATGTAGATCGTCAGCATTACC |
| No. 21) | AGACAATACTGCCGACACACATAACC |
| ODN-22 | GCATTCGAGCAACGTAAGATCCTGAACTGGCATGACA |
| (SEQ. ID. | GTGAGCTATGCCTCGCACTCTATCTACCTGGTATGAC |
| No. 22) | ACTGGCATGCTGTGAGAGCATATGT^{Me}C^{k} |
| ODN-23 | CTGCTCAGACGACGCGGCACGAGCGATAGTCT |
| (SEQ. ID. | |
| No. 23) | |
| splint | |
| ODN-24 | AGAATTCGACGACCAGGACATATGCTCTCACA |
| (SEQ. ID. | |
| No. 24) | |
| splint | |
| ODN-25 | GCATTCGAGCAACGTAAGATCCTGAACTGGCATGACA |
| (SEQ. ID. | GTGAGCTATGCCTCGCACTCTATCTACCTGGTATGAC |
| No. 25) | ACTGGCATGCTGTGAGAGCATATGT^{Me}CₜCTGGTCGTC |
| 300-mer | GAATTCTAGTAGATGTCTACATGTACAGATGTCGATA |
| | CGCCAGTACGCGCTAGGATCACATACGCGCAGACGTA |
| | TAGACTATCGCTCGTG^{Me}CₜCGCGTCGTCTGAGCAGTCT |
| | GATCGTGTCTGAGTACGCATGATCTGGATGTGTGATG |
| | TAGATCGTCAGCATTACCAGACAATACTGCCGACACA |
| | CATAACC |
| ODN-26 | GCATTCGAGCAACGTAAG short primer 1 |
| (SEQ. ID. | |
| No. 26) | |
| (P) | |
| ODN-27 | GGTTATGTGTGTCGGCAG short primer 2 |
| (SEQ. ID. | |
| No. 27) | |
| (P) | |
| ODN-28 | CGCGCCATGGGCATTCGAGCAACGTAAG long |
| (SEQ. ID. | primer 1 |
| No. 28) | |
| (P) | |
| ODN-29 | CGCGCTCGAGGGTTATGTGTGTCGGCAG long |
| (SEQ. ID. | primer 2 |
| No. 29) | |
| (P) | |
| ODN-30 | ^{z}TCGGTCGTCGAATTCTAGTAGATGTCFACATGTACAG |
| (SEQ. ID. | ATGTCGATACGCCAGTACGCGCTAGGATCACATACGC |
| No. 30) | GCAGACGTATAGACTATCGCTCGTG^{Me}C^{k} |
| (P) | |
| ODN-31 | CGGTCGTCGAATTCTAGTAGATGTCFACATGTACAGA |
| (SEQ. ID. | TGTCGATACGCCAGTACGCGCTAGGATCACATACGCG |
| No. 31) | CAGACGTATAGACTATCGCTCGTG^{Me}CₜT |
| | Cyclic oligonucleotide, same sequence |
| | as linear ODN-30 (SEQ. ID. No. 30) |
| | with ^{Me}CₜT triazole linkage |
| ODN-32 | GAGCGATAGTCTATACGT |
| (SEQ. ID. | |
| No. 32) | |
| (P) | |
| ODN-33 | TCGTCGAATTCTAGTAGA |
| (SEQ. ID. | |
| No. 33) | |
| (P) | |
| ODN-32a | GAGCGATAGTCTATACxGxT |
| (SEQ. ID. | |
| No. 32a) | |
| (P) | |
| ODN-33a | TCGTCGAATTCTAGTAxGxA |
| (SEQ. ID. | |
| No. 33a) | |
| (P) | |
| ODN-34 | tmsGCGCGTACFGGCGFATCGP |
| (SEQ. ID. | |
| No. 34) | |
| (HyBe) | |
| ODN-35 | GTTGTTAGTACTCA^{Me}C^{k} |
| (SEQ. ID. | |
| No. 35) | |
| ODN-36 | ^{z}CAGTCACAGAAAAGC |
| (SEQ. ID. | |
| No. 36) | |
| ODN-37 | GTTGTTCGATCGTTGTCAGAAGTAAGTTGG^{Me}C^{k} |
| (SEQ. ID. | |
| No. 37) | |
| ODN-38 | ^{z}CGCAGTGTTATCACT |
| (SEQ. ID. | |
| No. 38) | |
| ODN-39 | GTTGTTAGTACTCA^{Me}CₜCAGTCACAGAAAAGC |
| (SEQ. ID. | BLA ScaI forward tz |
| No. 39) | |
| (P) | |
| ODN-40 | GTTGTTAGTACTCACCAGTCACAGAAAAGC |
| (SEQ. ID. | BLA ScaI forward |
| No. 40) | |
| (P) | |
| ODN-41 | GTTGTTCGATCGTTGTCAGAAGTAAGTTGG^{Me}CₜCGCA |
| (SEQ. ID. | GTGTTATCACT BLA PvuI reverse tz |
| No. 41) | |
| (P) | |
| ODN-42 | GTTGTTCGATCGTTGTCAGAAGTAAGTTGGCCGCAGT |
| (SEQ. ID. | GTTATCACT BLA PvuI reverse |
| No. 42) | |
| (P) | |
| ODN-43 | CTGTGACTGGTGAGTACT3 |
| (SEQ. ID. | |
| No. 43) | |
| splint | |
| ODN-44 | AACACTGCGGCCAACTTA3 |
| (SEQ. ID. | |
| No. 44) | |
| splint | |
| ODN-45 | 5GGTTATGTGTGTCGGCAG |
| (SEQ. ID. | |
| No. 45) | |
| (P) | |
| ODN-46 | GCATTCGAGCAACGTAAGATCGCCAGCACACAATCTC |
| (SEQ. ID. | ACACTCTGGAATTCACACTGACAATACCAATACACAC |
| No. 46) | AGCCGTC Linear unmodified 81-mer |
| ODN-47 | 5GACGGCTGTGTGTATTGG |
| (SEQ. ID. | |
| No. 47) | |
| (P) | |
| ODN-48 | PhTCGGTCGTCGAATTCTAGTAGATGTCTACATGTACA |
| (SEQ. ID. | GATGTCGATACGCCAGTACGCGCTAGGATCACATACG |
| No. 48) | CGCAGACGTATAGACTATCGCTCGTGC |
| linear | |
| ODN-49 | PhTCGGTCGTCGAATTCTAGTAGATGTCTACATGTACA |
| (SEQ. ID. | GATGTCGATACGCCAGTACGCGCTAGGATCACATACG |
| No. 49) | CGCAGACGTATAGACTATCGCTCGTGC |
| cyclic | Same sequence as ODN-48 (SEQ. ID. No. |
| | 48) but cyclic with phosphate linkage |
| ODN-50 | GAATTCGACGACCGAGCACGAGCGATAGTC |
| (SEQ. ID. | |
| No. 50) | |
| splint | |

^{z}= 5′-azide,
^{k}= 3′-propargyl,
ₜ= triazole linkage,
F = fluorescein dT,
3 = 3′-fluorescein C7,
5 = 5′-fluorescein C6,
P = propanol,
Ph = 5′-phosphate (Link Technologies),
x = phosphothioate,
Tms = trimethoxystilbene.
HyBe = HyBeacon.
Lower case sequences are oligonucleotides made from 5′ to 3′ using reverse phosphoramidite monomers

### PCR and Sequencing of Triazole DNA Templates

### PCR Using GoTaq DNA Polymerase

PCR products from 81-mer, 210-mer and 300-mer templates were generated using GoTaq DNA polymerase (available from Promega) with 4 μL of 5× buffer (green buffer) in a total reaction volume of 20 μL with 5 ng of the DNA template, 0.5 μM of each primer, 0.2 mM dNTP and 0.5 unit of GoTaq. The reaction mixture was loaded onto a 2% agarose gel in 1×TBE buffer. PCR cycling conditions: 95° C. (initial denaturation) for 2 min then 25 cycles of 95° C. (denaturation) for 15 sec, 54° C. (annealing) for 20 sec and 72° C. (extension) for 30 sec. 5× Promega green PCR buffer was provided with the enzyme (containing Tris.HCl, KCl, 7.5 mM MgCl₂, pH 8.5) to give a final Mg²⁺ concentration of 1.5 mM.

### PCR Using Pfu DNA Polymerase

PCR product from ODN-08 (SEQ. ID. No. 08) (81-mer CC template) was generated using 2 μL of 10× buffer in a total reaction volume of 20 μL with 5 ng of the DNA template, 0.5 μM of each primer, 0.2 mM dNTP and 1.0 unit of Pfu DNA polymerase. (10× reaction buffer=200 mM Tris-HCl (pH 8.8), 100 mM KCl, 100 mM (NH₄)₂SO₄, 20 mM MgSO₄, 1.0% Triton® X-100 and 1 mg/ml nuclease-free BSA). PCR cycling conditions: 95° C. (initial denaturation) for 2 min then 25 cycles of 95° C. for 15 sec, 54° C. for 20 sec and 72° C. for 30 sec. This was followed by one cycle of 72° C. for 2 min.

Sequencing of Clones from the PCR Product of 81-mer, 210-mer and 300-mer Triazole Templates.

The PCR products were prepared as above using GoTaq or Pfu DNA polymerase and purified on a 2% agarose gel followed by extraction using QIAquick Gel Extraction kit Cat. No. 28704. The purified PCR products were then cloned and sequenced by the automated fluorescent Sanger method: 10 clones for ODN-25 (SEQ. ID. No. 25) (300-mer with two ^{Me}CₜC linkages), 50 clones for ODN-19 (SEQ. ID. No. 19) (210-mer with two ^{Me}CₜT linkages), 40 clones for ODN-08 (SEQ. ID. No. 08) (81-mer with ^{Me}CₜC linkage), 17 clones for ODN-07 (SEQ. ID. No. 07) (81-mer with TₜC linkage) and 17 clones for ODN-06 (SEQ. ID. No. 06) (81-mer with TₜT linkage). ODN-08 (SEQ. ID. No. 08) (81-mer with ^{Me}CₜC) was amplified using both GoTaq and Pfu DNA polymerases, and 20 clones of each were sequenced. The polymerases read the sequence around the triazole linkages correctly for all 134 sequences.

PCR of BLA Fragment with Click Primers

The region between the ScaI and PvuI sites of BLA was amplified by PCR with GoTaq DNA polymerase using the click-linked oligonucleotides ODN-39 (SEQ. ID. No. 39) and ODN-41 (SEQ. ID. No. 41), 10 μl of 5× buffer in a total reaction volume of 50 μL with 1 ng of the DNA template, 1 μM of each primer, 0.2 mM dNTP and 1 unit of GoTaq. The reaction was repeated with normal oligonucleotides (no triazole linker). The reaction mixtures were loaded onto a 2% agarose gel in 1×TAE buffer; both reactions gave products of identical size. PCR cycling conditions were: 94° C. (initial denaturation) for 1.5 min then 35 cycles of 94° C. (denaturation) for 30 sec, 46.5° C. (annealing) for 30 sec and 72° C. (extension) for 30 sec. The reaction was held at 72° C. for 5 minutes after the 35 cycles.

### Restriction Digestion of PCR Product and Vector

The PCR products were digested with Seal HF and PvuI restriction endonucleases (NEB, Cat. No. R3122 and R0150) according to the manufacturer's protocol and was purified using QIAquick PCR purification kit (QIAGEN, Cat. No. 28106). The Luciferase T7 control plasmid (Promega, Cat. No. L4821) was also digested with Seal HF and PvuI, and treated with thermosensitive alkaline phosphatase (Promega, Cat. No. M9910) to remove the 5′-phosphate groups from the linearized plasmid DNA, thus preventing recircularisation during ligation. The linear plasmid was gel-purified using QIAquick gel extraction kit (QIAGEN Cat. No. 28706) to remove the undigested plasmid and the excised fragment.

Ligation Reactions and Transformation into E. coli

The digested PCR products (triazole and normal) and linearized plasmid were ligated for 16 hr at 15° C. (total volume 10 μL, 1:3 vector:insert ratio) using T4 DNA ligase (Promega, Cat. No. M1801). Negative control ligations were set up as above, using water instead of insert. 5 μL of each ligation mixture was transformed into chemically competent E. coli (NEB 5α, NEB, Cat. No. C2992H) using the standard protocol. Transformants were recovered in 895 μL of SOC at 37° C. with shaking for one hour. 100 μL of each recovery solution was spread onto LB agar plates and incubated at 37° C. overnight. Colonies were counted using a Gel Doc XR+ system and Quantity One Software (both from Bio-Rad Laboratories). The above procedure was repeated for the UvrB deficient E. coli strain (JW0762-2, CGSC, Cat. No. 8819) which was supplied by the Coli Genetic Stock Center (CGSC) at Yale University.

### Sequencing of the BLA Gene

50 Colonies were picked from plates containing the plasmids with the triazole DNA insert in its BLA gene and 50 were picked from the positive control plates (normal BLA gene). The colonies were grown overnight in LB and the plasmids from each culture were isolated using QIAprep Spin miniprep kit (QIAGEN, Cat. No. 27106). They were then sequenced by the automated fluorescent Sanger method. Mutations were not observed in the region between the ScaI and PvuI sites in any of the plasmids. For the experiment on DNA repair, 21 colonies were sequenced from repair-deficient E. coli strain JW0762-2 and all sequences were found to be correct.

All publications and patents mentioned in the above specification are herein incorporated by reference. Various modifications and variations of the described methods, systems and products of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, genetics, chemistry or related fields are intended to be within the scope of the following claims.

1. A method for ligating one or more oligonucleotides, said method comprising reacting at least one alkyne group with at least one azide group to form at least one triazole phosphodiester mimic, wherein said reaction is selected from the following reaction schemes or an RNA equivalent thereof:

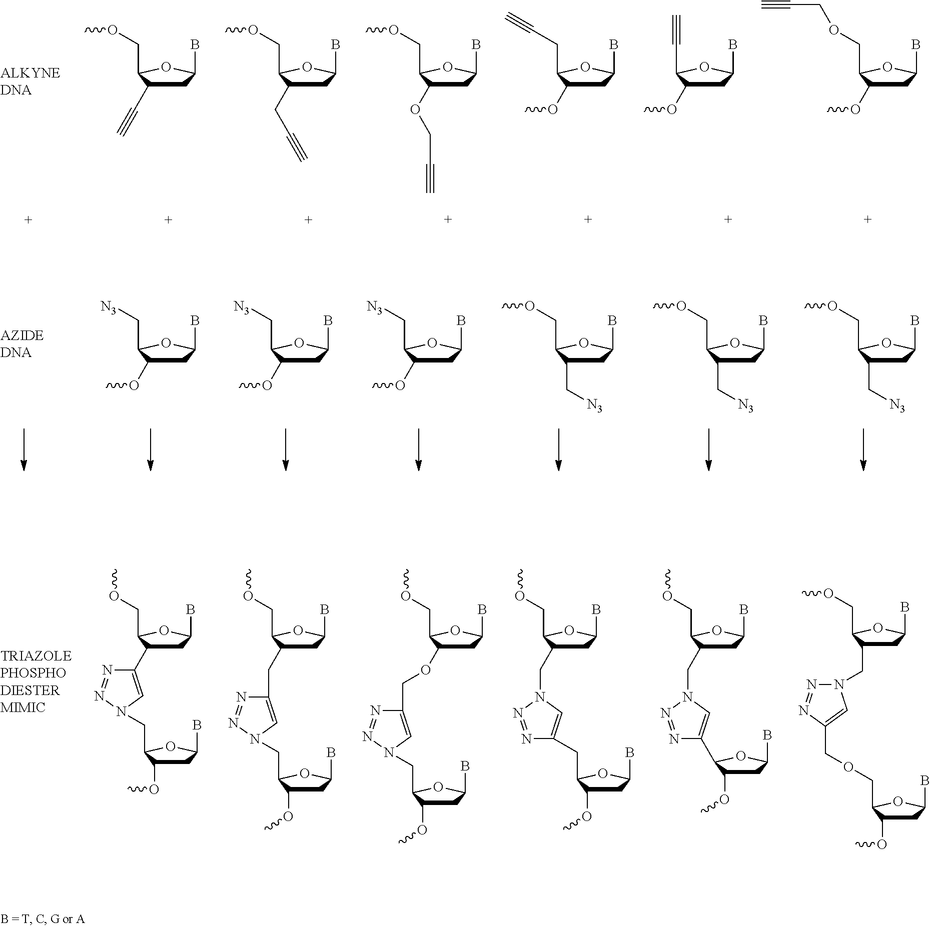

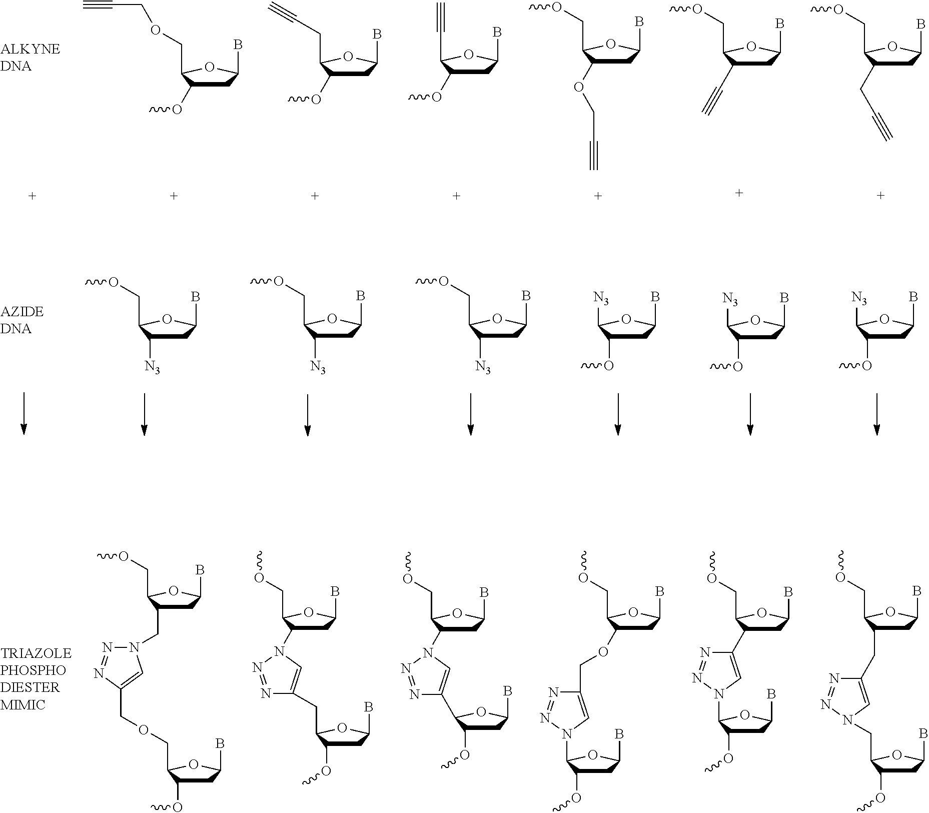

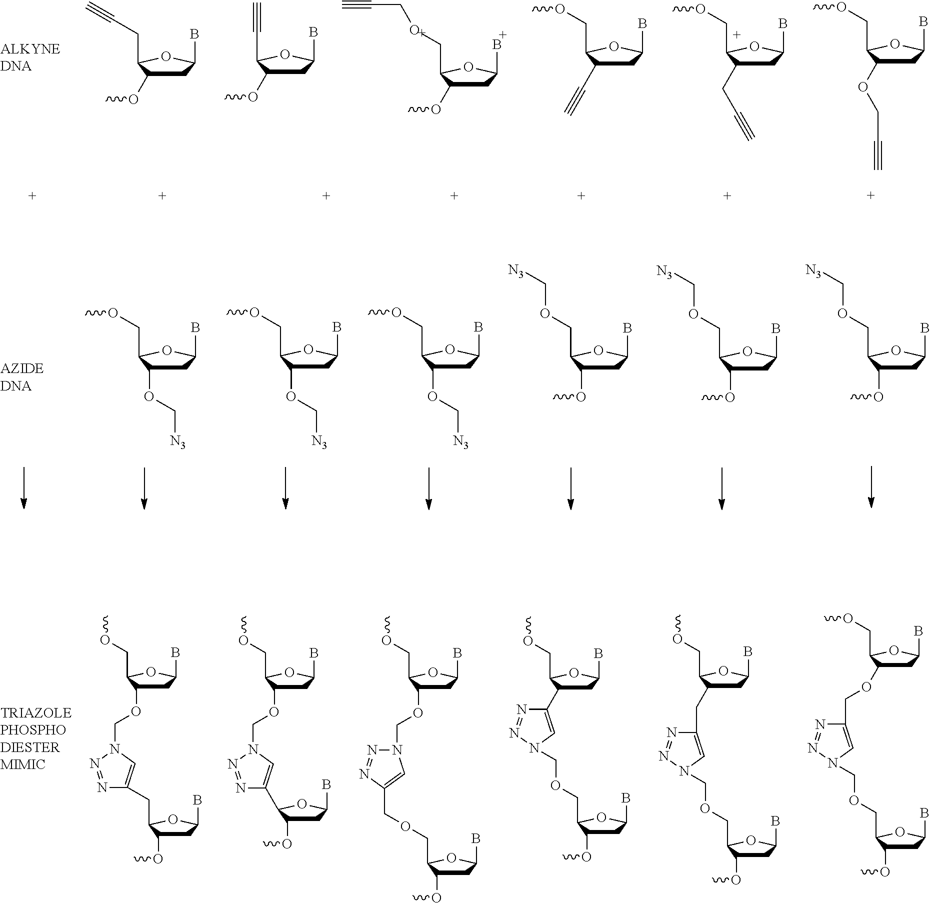

2. A method according to claim 1 wherein two or more oligonucleotides are ligated together to form one or more triazole phosphodiester mimics.

3. A method according to claim 1 wherein said at least one triazole phosphodiester mimic can be read through accurately by a DNA polymerase and/or an RNA polymerase.

4. A method according to claim 1 wherein said reaction follows the reaction scheme below or an RNA equivalent thereof:

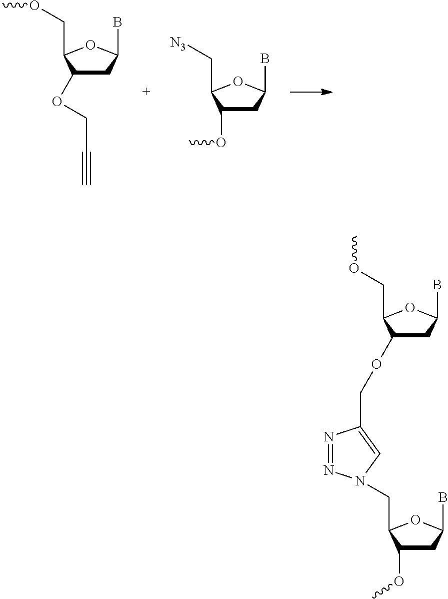

5. A method according to claim 1 wherein an alkyne group at the 5′ or 3′ end of one oligonucleotide is reacted with an azide group is at the 5′ or 3′ end of a second oligonucleotide to form at least one triazole phosphodiester mimic.

6. A method according to claim 1 wherein an alkyne group at the 3′ end of one oligonucleotide is reacted with an azide group is at the 5′ end of a second oligonucleotide to form at least one triazole phosphodiester mimic.

7. A method according to claim 1 wherein a double stranded oligonucleotide is ligated to a second double stranded oligonucleotide to form a double stranded oligonucleotide with at least one triazole phosphodiester mimic in each ligated strand.

8. A method according to claim 5 1 wherein a single or double stranded oligonucleotide is circularized by reacting at least one alkyne group at one end of the oligonucleotide with at least one azide group at the other end of the oligonucleotide to form at least one triazole phosphodiester mimic in each cyclized strand.

9. A method according to claim 1 wherein the reaction of at least one alkyne group with at least one azide group is carried out on a solid phase.

10. A method according to claim 1 wherein the reaction of at least one alkyne group with at least one azide group is carried out under templated conditions.

11. A method according to claim 1 wherein the reaction of at least one alkyne group with at least one azide group is carried out under templated conditions and the template is obtained or obtainable by the method according to claim 1.

12. A method according to claim 1 wherein the reaction of at least one alkyne group with at least one azide group is carried out under templated conditions and the template is a cyclic single-stranded oligonucleotide and wherein a double-stranded helical oligonucleotide catenane is prepared.

13. A method according to claim 1 wherein the reaction of at least one alkyne group with at least one azide group is carried out under templated conditions using a single stranded circularized oligonucleotide as a template for the cyclization of a second linear complementary oligonucleotide by reacting an alkyne group at one end of the linear oligonucleotide with an azide group at the other 5 end of the linear oligonucleotide and wherein a double stranded DNA catenane which contains at least one triazole backbone linkage that can be read through correctly by DNA and/or RNA polymerases is produced.

14. A method according to claim 1 wherein the step of reacting at least one alkyne group with at least one azide group to form at least one triazole phosphodiester mimic is repeated more than once to form an oligonucleotide comprising more than one triazole phosphodiester mimic.

15. A method according to claim 1 for ligating more than one oligonucleotide wherein at least one oligonucleotide is DNA and at least one oligonucleotide is RNA.

16. A method according to claim 1 wherein at least one oligonucleotide comprises at least one DNA analogue and/or at least one RNA analogue and/or at least one modified nucleotide and/or at least one labelled nucleotide.

17. A method according to claim 1 wherein the reaction is catalyzed by Copper (I).

18. An oligonucleotide construct obtainable or obtained by a method according to claim 1.

19. An oligonucleotide construct comprising at least one triazole phosphodiester mimic having a structure selected from the following or an RNA equivalent thereof:
TRIAZOLE PHOSPHO DIESTER MIMICS B=T, C, G or A

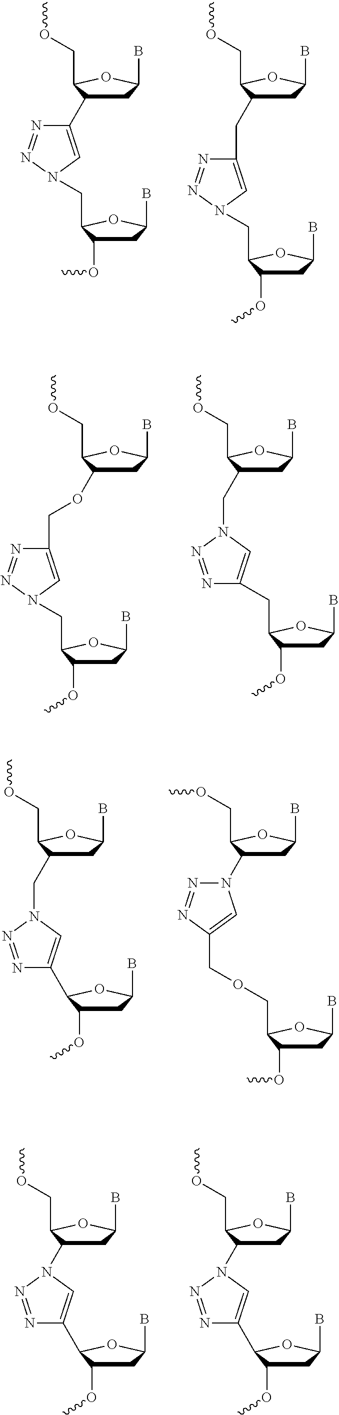

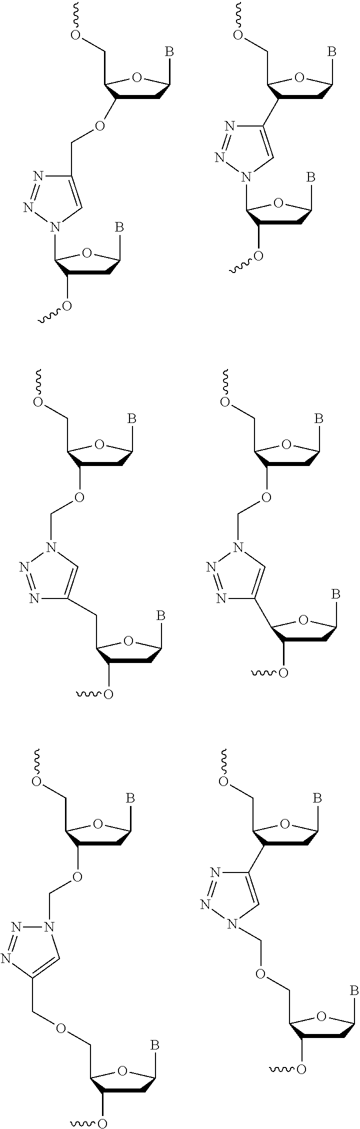

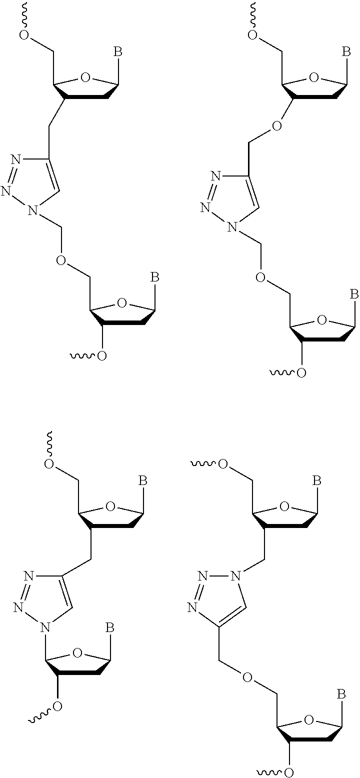

20. An oligonucleotide construct according to claim 19 comprising at least two triazole phosphodiester mimics each having a structure selected from the structures described in claim 19 or an RNA equivalent thereof.

21. An oligonucleotide construct according to claim 19 comprising at least one triazole phosphodiester mimic having the following structure or an RNA equivalent thereof:

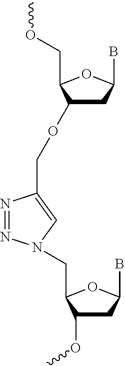

22. An oligonucleotide construct according to claim 19 wherein said at least one triazole phosphodiester mimic can be read through accurately by a DNA polymerase and/or an RNA polymerase.

23. An oligonucleotide construct according to claim 19 comprising a double stranded oligonucleotide.

24. An oligonucleotide construct according to claim 19 comprising a single stranded oligonucleotide.

25. An oligonucleotide construct according to claim 19 comprising a circularized oligonucleotide.

26. An oligonucleotide comprising one or more alkyne groups and/or one or more azide groups for use in a method according to claim 1.


SEQUENCE LISTING (53 sequences)

SEQ ID NO: 1 - DNA, 58 bp, Artificial Sequence
  Oligonucleotide-1 beginning with a ({circumflex over ())}{circumflex over (})}z)T base unit, wherein z is a 5′-azide terminal group
taccacacaatctcacactctggaattcacactgacaatactgccgacacacataacc

SEQ ID NO: 2 - DNA, 58 bp, Artificial Sequence
  Oligonucleotide-2 beginning with a ({circumflex over ())}{circumflex over (})}z)C base unit, wherein z is a 5′-azide terminal group
cagcacacaatctcacactctggaattcacactgacaatactgccgacacacataacc

SEQ ID NO: 3 - DNA, 23 bp, Artificial Sequence
  Oligonucleotide-3 with ({circumflex over ())}{circumflex over (})}Me)C({circumflex over ())}{circumflex over (})}k) base unit after nucelotide number 22, wherein k is a 3′-propargyl terminal group
gcattcgagcaacgtaagatcgc

SEQ ID NO: 4 - DNA, 23 bp, Artificial Sequence
  Oligonucleotide-4 ending with a t({circumflex over ())}{circumflex over (})}k) base unit, wherein k is a 3′-propargyl terminal group
gcattcgagcaacgtaagatcct

SEQ ID NO: 5 - DNA, 23 bp, Artificial Sequence
  Oligonucleotide-5 ending with a t({circumflex over ())}{circumflex over (})}k) base unit, wherein k is a 3′-propargyl terminal group
gcattcgagcaacgtaagatcgt

SEQ ID NO: 6 - DNA, 81 bp, Artificial Sequence
  Oligonucleotide-6 is a 81-mer with T({circumflex over ())}{circumflex over (})}t)T base unit after nucleotide number 22
gcattcgagcaacgtaagatccttaccacacaatctcacactctggaattcacactgacaatactgccgacacacataacc

SEQ ID NO: 7 - DNA, 81 bp, Artificial Sequence
  Oligonucleotide-7 is a 81-mer with T({circumflex over ())}{circumflex over (})}t)C base unit after nucleotide number 22
gcattcgagcaacgtaagatcgtcagcacacaatctcacactctggaattcacactgacaatactgccgacacacataacc

SEQ ID NO: 8 - DNA, 81 bp, Artificial Sequence
  Oligonucleotide-8 is a 81-mer with ({circumflex over ())}{circumflex over (})}Me)C({circumflex over ())}{circumflex over (})}t)C base unit after nucleotide number 22
gcattcgagcaacgtaagatcgccagcacacaatctcacactctggaattcacactgacaatactgccgacacacataacc

SEQ ID NO: 9 - DNA, 11 bp, Artificial Sequence
  Oligonucleotide-9 with ({circumflex over ())}{circumflex over (})}Me)C({circumflex over ())}{circumflex over (})}k) base unit after nucelotide number 10, wherein k is a 3′-propargyl terminal group
gcattcatgtc

SEQ ID NO: 10 - DNA, 11 bp, Artificial Sequence
  Oligonucleotide-10 beginning with a ({circumflex over ())}{circumflex over (})}z)C base unit, wherein z is a 5′-azide terminal group; and having a ({circumflex over ())}{circumflex over (})}Me)C({circumflex over ())}{circumflex over (})}k) base unit after nucelotide number 10, wherein k is a 3′-propargyl terminal group
ctggtccgtgc

SEQ ID NO: 11 - DNA, 11 bp, Artificial Sequence
  Oligonucleotide-11 beginning with a ({circumflex over ())}{circumflex over (})}z)C base unit, wherein z is a 5′-azide terminal group
cgcgtctaacc

SEQ ID NO: 12 - DNA, 33 bp, Artificial Sequence
  Oligonucleotide-12 is a 33-mer with ({circumflex over ())}{circumflex over (})}Me)C({circumflex over ())}{circumflex over (})}t)C base units after nucleotide numbers 10, 21
gcattcatgtcctggtccgtgccgcgtctaacc

SEQ ID NO: 13 - DNA, 41 bp, Artificial Sequence
  Oligonucleotide-13 begnning with 5T base unit, wherein 5 is a 5′-fluorescein C6 terminal group
ttttggttagacgcggcacggaccaggacatgaatgctttt

SEQ ID NO: 14 - DNA, 70 bp, Artificial Sequence
  Oligonucleotide-14 beginning with a ({circumflex over ())}{circumflex over (})}z)C group, wherein z is a 5′-azide terminal group; and having a ({circumflex over ())}{circumflex over (})}Me)C({circumflex over ())}{circumflex over (})}k) base unit after nucelotide number 69, wherein k is a 3′-propargyl terminal group
tcggtcgtcgaattctagtagatgtctacatgtacaacatacgcgcagacgtatagactatcgctcgtgc

SEQ ID NO: 15 - DNA, 70 bp, Artificial Sequence; identical to SEQ ID NO: 14
  Oligonucleotide-14a is a cyclic construct of Oligonucleotide-14 with ({circumflex over ())}{circumflex over (})}Me)C({circumflex over ())}{circumflex over (})}t)T linkage

SEQ ID NO: 16 - DNA, 70 bp, Artificial Sequence
  Oligonucleotide-14a is a cyclic construct of Oligonucleotide-14 with ({circumflex over ())}{circumflex over (})}Me)C({circumflex over ())}{circumflex over (})}t)T linkage
gcattcgagcaacgtaagatcctgaactggcatgacggtatgacactggcatgctgtgagagcatatgtc

SEQ ID NO: 17 - DNA, 70 bp, Artificial Sequence
  Oligonucleotide-16 with ({circumflex over ())}{circumflex over (})}Me)C({circumflex over ())}{circumflex over (})}k) base unit after nucelotide number 69, wherein k is a 3′-propargyl terminal group
tgcgtcgtctgagcagtctgatcgtgtctgagtacggcattaccagacaatactgccgacacacataacc

SEQ ID NO: 18 - DNA, 40 bp, Artificial Sequence
  Oligonucleotide-17 is a splint oligonuceotide complimentary to the ends of two oligonuceotide to be ligated
tactagaattcgacgaccgagacatatgctctcacagcat

SEQ ID NO: 19 - DNA, 40 bp, Artificial Sequence
  Oligonucleotide-18 is a splint oligonuceotide complimentary to the ends of two oligonuceotide to be ligated
cagactgctcagacgacgcagcacgagcgatagtctatac

SEQ ID NO: 20 - DNA, 206 bp, Artificial Sequence
  Oligonucleotide-19 is a 210-mer with ({circumflex over ())}{circumflex over (})}Me)C({circumflex over ())}{circumflex over (})}t)C base units after nucleotide numbers 69, 139
gcattcgagcaacgtaagatcctgaactggcatgacggtatgacactggcatgctgtgagagcatatgtcggtcgtcgaattctagtagatgtctacatgtacaacatacgcgcagacgtatagactatcgctcgtggcgtcgtctgagcagtctgatcgtgtctgagtacggcattaccagacaatactgccgacacacataacc

SEQ ID NO: 21 - DNA, 100 bp, Artificial Sequence
  Oligonucleotide-20 beginning with a ({circumflex over ())}{circumflex over (})}z)C base unit, wherein z is a 5′-azide terminal group; and having a ({circumflex over ())}{circumflex over (})}Me)C({circumflex over ())}{circumflex over (})}k) base unit after nucelotide number 99, wherein k is a 3′-propargyl terminal group
ctggtcgtcgaattctagtagatgtctacatgtacagatgtcgatacgccagtacgcgctaggatcacatacgcgcagacgtatagactatcgctcgtgc

SEQ ID NO: 22 - DNA, 100 bp, Artificial Sequence
  Oligonucleotide-21 beginning with a ({circumflex over ())}{circumflex over (})}z)C group, wherein z is a 5′-azide terminal group
cgcgtcgtctgagcagtctgatcgtgtctgagtacgcatgatctggatgtgtgatgtagatcgtcagcattaccagacaatactgccgacacacataacc

SEQ ID NO: 23 - DNA, 100 bp, Artificial Sequence
  Oligonucleotide-22 with ({circumflex over ())}{circumflex over (})}Me)C({circumflex over ())}{circumflex over (})}k) base unit after nucelotide number 99, wherein k is a 3′-propargyl terminal group
gcattcgagcaacgtaagatcctgaactggcatgacagtgagctatgcctcgcactctatctacctggtatgacactggcatgctgtgagagcatatgtc

SEQ ID NO: 24 - DNA, 32 bp, Artificial Sequence
  Oligonucleotide-23 is a splint oligonuceotide complimentary to the ends of two oligonuceotide to be ligated
ctgctcagacgacgcggcacgagcgatagtct

SEQ ID NO: 25 - DNA, 32 bp, Artificial Sequence
  Oligonucleotide-24 is a splint oligonuceotide complimentary to the ends of two oligonuceotide to be ligated
agaattcgacgaccaggacatatgctctcaca

SEQ ID NO: 26 - DNA, 300 bp, Artificial Sequence
  Oligonucleotide-25 is a 300-mer with ({circumflex over ())}{circumflex over (})}Me)C({circumflex over ())}{circumflex over (})}t)C base units after nucleotide numbers 99, 199
gcattcgagcaacgtaagatcctgaactggcatgacagtgagctatgcctcgcactctatctacctggtatgacactggcatgctgtgagagcatatgtcctggtcgtcgaattctagtagatgtctacatgtacagatgtcgatacgccagtacgcgctaggatcacatacgcgcagacgtatagactatcgctcgtgccgcgtcgtctgagcagtctgatcgtgtctgagtacgcatgatctggatgtgtgatgtagatcgtcagcattaccagacaatactgccgacacacataacc

SEQ ID NO: 27 - DNA, 18 bp, Artificial Sequence
  Oligonucleotide-26 is short primer for PCR amplification for 210-mer and 300-mer click ligated triazole DNA templates
gcattcgagcaacgtaag

SEQ ID NO: 28 - DNA, 18 bp, Artificial Sequence
  Oligonucleotide-27 is short primer for PCR amplification for 210-mer and 300-mer click ligated triazole DNA templates
ggttatgtgtgtcggcag

SEQ ID NO: 29 - DNA, 28 bp, Artificial Sequence
  Oligonucleotide-28 is long primer for PCR amplification for 210-mer and 300-mer click ligated triazole DNA templates
cgcgccatgggcattcgagcaacgtaag

SEQ ID NO: 30 - DNA, 28 bp, Artificial Sequence
  Oligonucleotide-29 is long primer for PCR amplification for 210-mer and 300-mer click ligated triazole DNA templates
cgcgctcgagggttatgtgtgtcggcag

SEQ ID NO: 31 - DNA, 99 bp, Artificial Sequence
  Oligonucleotide-30 beginning with a ({circumflex over ())}{circumflex over (})}z)T base unit, wherein z is a 5′-azide terminal group; and having a ({circumflex over ())}{circumflex over (})}Me)C({circumflex over ())}{circumflex over (})}k) base unit after nucelotide number 99, wherein k is a 3′-propargyl terminal
tcggtcgtcgaattctagtagatgtcacatgtacagatgtcgatacgccagtacgcgctaggatcacatacgcgcagacgtatagactatcgctcgtgc

SEQ ID NO: 32 - DNA, 98 bp, Artificial Sequence
  Oligonucleotide-31 is a cyclic oligonucleotide of Oligonucleotide-30 with ({circumflex over ())}{circumflex over (})}Me)C({circumflex over ())}{circumflex over (})}t)T triazole linkage
tcggtcgtcgaattctagtagatgtcacatgtacagatgtcgatacgccagtacgcgctaggatcacatacgcgcagacgtatagactatcgctcgtg

SEQ ID NO: 33 - DNA, 18 bp, Artificial Sequence
  Oligonucleotide-32 is a primer for PCR amplification
gagcgatagtctatacgt

SEQ ID NO: 34 - DNA, 18 bp, Artificial Sequence
  Oligonucleotide-33 is a primer for PCR amplification
tcgtcgaattctagtaga

SEQ ID NO: 35 - DNA, 18 bp, Artificial Sequence
  Oligonucleotide-32a is a primer for PCR amplification having phosphothioate groups after nucleotide numbers 16, 17
gagcgatagtctatacgt

SEQ ID NO: 36 - DNA, 18 bp, Artificial Sequence
  Oligonucleotide-33a is a primer for PCR amplification having phosphothioate groups after nucleotide numbers 16, 17
tcgtcgaattctagtaga

SEQ ID NO: 37 - DNA, 16 bp, Artificial Sequence
  Oligonucleotide-34 beginning with a 5′-trimethoxystilbene terminal group and ending with a 3′-propanol terminal group; and having fluorescein dT groups after nucleotide numbers 8, 12
gcgcgtacggcgatcg

SEQ ID NO: 38 - DNA, 15 bp, Artificial Sequence
  Oligonucleotide-35 with ({circumflex over ())}{circumflex over (})}Me)C({circumflex over ())}{circumflex over (})}k) base unit after nucelotide number 14, wherein k is a 3′-propargyl terminal group
gttgttagtactcac

SEQ ID NO: 39 - DNA, 15 bp, Artificial Sequence
  Oligonucleotide-36 beginning with a ({circumflex over ())}{circumflex over (})}z)C base unit, wherein z is a 5′-azide terminal group
cagtcacagaaaagc

SEQ ID NO: 40 - DNA, 31 bp, Artificial Sequence
  Oligonucleotide-37 with ({circumflex over ())}{circumflex over (})}Me)C({circumflex over ())}{circumflex over (})}k) base unit after nucelotide number 30, wherein k is a 3′-propargyl terminal group
gttgttcgatcgttgtcagaagtaagttggc

SEQ ID NO: 41 - DNA, 15 bp, Artificial Sequence
  Oligonucleotide-38 beginning with a ({circumflex over ())}{circumflex over (})}z)C base unit, wherein z is a 5′-azide terminal group
cgcagtgttatcact

SEQ ID NO: 42 - DNA, 30 bp, Artificial Sequence
  Oligonucleotide-39 is a primer for PCR amplification with a ({circumflex over ())}{circumflex over (})}Me)C({circumflex over ())}{circumflex over (})}t)C base unit after nucleotide number 14
gttgttagtactcaccagtcacagaaaagc

SEQ ID NO: 43 - DNA, 30 bp, Artificial Sequence; identical to SEQ ID NO: 42
  Oligonucleotide-40 is a primer for PCR amplification

SEQ ID NO: 44 - DNA, 46 bp, Artificial Sequence
  Oligonucleotide-41 is a primer for PCR amplification with a ({circumflex over ())}{circumflex over (})}Me)C({circumflex over ())}{circumflex over (})}t)C base unit after nucleotide number 30
gttgttcgatcgttgtcagaagtaagttggccgcagtgttatcact

SEQ ID NO: 45 - DNA, 46 bp, Artificial Sequence; identical to SEQ ID NO: 44
  Oligonucleotide-42 is a primer for PCR amplification

SEQ ID NO: 46 - DNA, 18 bp, Artificial Sequence
  Oligonucleotide-43 is a splint oligonucleotide having a 3′-fluorescein C7 terminal group and completementary to two ends of the oligonucleotides to be ligated together
ctgtgactggtgagtact

SEQ ID NO: 47 - DNA, 18 bp, Artificial Sequence
  Oligonucleotide-44 is a splint oligonucleotide having a 3′-fluorescein C7 terminal group and completementary to two ends of the oligonucleotides to be ligated together
aacactgcggccaactta

SEQ ID NO: 48 - DNA, 18 bp, Artificial Sequence
  Oligonucleotide-45 beginning with 5′-fluorescein C6 terminal group
ggttatgtgtgtcggcag

SEQ ID NO: 49 - DNA, 81 bp, Artificial Sequence
  Oligonucleotide-46 is a linear unmodified 81-mer
gcattcgagcaacgtaagatcgccagcacacaatctcacactctggaattcacactgacaataccaatacacacagccgtc

SEQ ID NO: 50 - DNA, 18 bp, Artificial Sequence
  Oligonucleotide-47 beginning with 5′-fluorescein C6 terminal group
gacggctgtgtgtattgg

SEQ ID NO: 51 - DNA, 100 bp, Artificial Sequence
  Oligonucleotide-48 beginning with a 5′-phosphate terminal group
tcggtcgtcgaattctagtagatgtctacatgtacagatgtcgatacgccagtacgcgctaggatcacatacgcgcagacgtatagactatcgctcgtgc

SEQ ID NO: 52 - DNA, 100 bp, Artificial Sequence; identical to SEQ ID NO: 51
  Oligonucleotide-49 is a cyclic oligonucleotide of oligonucleotide-48 with phosphate linkage

SEQ ID NO: 53 - DNA, 30 bp, Artificial Sequence
  Oligonucleotide-50 is a splint oligonuceotide complimentary to the ends of two oligonuceotide to be ligated
gaattcgacgaccgagcacgagcgatagtc